(19) United States
(12) Patent Application Publication
(10) Pub. No.: US 2009/0081725 A1
(43) Pub. Date: Mar. 26, 2009

(54) Compositions of pamps and Listeria monocytogenes and methods of use

(76) Inventors: Thomas J. Powell, Madison, CT (US); Ruslan M. Medzhitov, Branford, CT (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/498,926

(22) Filed: Aug. 3, 2006

Related U.S. Application Data
(63) Continuation-in-part of application No. PCT/US05/03367, filed on Feb. 4, 2005.
(60) Provisional application No. 60/542,739, filed on Feb. 6, 2004.

(51) Int. Cl. C12P 21/02; A61K 39/02; C07K 14/195; C07H 21/00; C12N 15/63; C12N 5/10

(52) U.S. Cl. 435/69.1; 424/234.1; 424/192.1; 424/190.1; 530/350; 536/23.7; 536/23.4; 435/320.1; 435/325

(57) ABSTRACT

A composition comprising a pathogen associated molecular pattern (PAMP) protein that activates toll-like receptor 2 (TLR2) or toll-like receptor 5 (TLR5) signaling and at least two distinct antigens of Listeria monocytogenes. Amino acids, nucleotides, vectors, cell lines, and the methods for production and use of the compositions are provided.

### RELATED APPLICATION

This application is a continuation-in-part of International Application No. PCT/US2005/003367, which designated the United States and was filed on Feb. 4, 2005, published in English, which claims the benefit of U.S. Provisional Application No. 60/542,739, filed on Feb. 6, 2004. The entire teachings of the above application are incorporated herein by reference.

### BACKGROUND OF THE INVENTION

### Listeria Pathogens

Listeria monocytogenes is a highly virulent and prevalent food-borne gram-positive bacillus that causes gastroenteritis in otherwise healthy patients (Wing et al., J. Infect. Dis. 2002, 185, 1: S18-24), and more severe complications in immunocompromised patients, including meningitis, encephalitis, bacteremia and morbidity (Crum, N. F., Curr. Gastroenterol. Rep. 2002, 4:287-296; Frye et al., Clin. Infect. Dis. 2002, 35:943-949). In the United States alone it is estimated that each year 2,500 people become seriously ill with L. monocytogenes, resulting in 500 deaths (Centers for Disease Control and Prevention Technical information 2002, (http://www.cdc.gov/ncidQd/dbmd/diseaseinfo/listeriosis_t.htm). In pregnant women, L. monocytogenes infection can have devastating results, including miscarriage rates of 25-45% (Wing et al, op cit.). An analysis of food-borne illness in 1998 revealed that L. monocytogenes infections resulted in the highest hospitalization rate (98%) and the highest fatality rate (15%) among bacterial pathogens, surpassing even E. coli (CDC, 1999). L. monocytogenes has been isolated from numerous points in the food preparation and distribution chain, including produce farms and packing plants (Prazak et al., J. Food Prot. 2002, 65:1728-1734), dairy farms and storage tanks (Waak et al., Appl. Environ. Microbiol. 2002 68:3366-3370), and cooked delicatessen meat (Frye et al., op cit.). Control of L. monocytogenes is additionally complicated by the pathogen's ability to grow at temperatures as low as 4° C., which inhibits the growth of most other bacterial pathogens (Gellin et al., JAMA 1989, 161:313-1320). The ease of dissemination of L. monocytogenes in contaminated food products presents the possibility of a widespread poisoning of the public food and/or water supply. Although the usual treatment for infection is antibiotics (Crum, N. F., Curr. Gastroenterol. Repl. 2002, 4:287-296), there is a need for efficacious, cost-effective preventive strategies, including vaccines, for Listeria contamination and infection.

In vivo models have identified roles for both T and B cells in response to L. monocytogenes, with protective immunity attributed primarily to CD8 cytotoxic T cells (CTL) (Kerksiek et al., Current Opinion in Immunology 1999, 11:400-405). Studies during the past several years have led to the identification of several immunodominant L. monocytogenes epitopes recognized by CD4 and CD8 T cells. In BALB/c mice, several peptides have been identified including the H-2K^{d} restricted epitopes LLO₉₁₋₉₉ and p60₂₁₇₋₂₂₅ (Pamer et al., Nature 1994, 353: 852-854). The vaccine potential for such peptides is supported by studies demonstrating that the transfer of LLO₉₁₋₉₉-specific CTL into naïve hosts conveys protection to a lethal challenge with L. monocytogenes (Harty et al., J. Exp. Med. 1992, 175:1531-1538). Notably, CTL stimulated with LLO₉₁₋₉₉ peptide alone provide protection only when the bacterial challenge is administered within a week of CTL transfer. However, the limited period of the protective effect can be augmented when the antigen is delivered in the presence of stimulatory factors including heat killed bacteria, anti-CD40 antibody, IL-12 or liposomes (Xiong et al., Immunology 1998. 94:14-21; Tuma et al., J. Clin. Investigation 2002, 110:1493-1501; Miller et al., Annals of the New York Academy of Sciences 1997, 797:207-227; Lipford et al., Immunology Letters 1994, 40:101-104). Collectively these and other studies demonstrate immune responses to specific listerial antigens can be enhanced when delivered in the context of adjuvants that provide additional stimulatory signals during lymphocyte activation. Recent advancements in our understanding of the innate immune system now provide an ideal opportunity to generate more effective vaccines against L. monocytogenes incorporating novel adjuvants of defined specificity and biological activity.

### Innate Immunity

Multicellular organisms have developed two general systems of immunity to infectious agents. The two systems are innate or natural immunity (referred to herein as “innate immunity”) and adaptive (acquired) or specific immunity. The major difference between the two systems is the mechanism by which they recognize infectious agents.

The innate immune system uses a set of germline-encoded receptors for the recognition of conserved molecular patterns present in microorganisms. These molecular patterns occur in certain constituents of microorganisms including: lipopolysaccharides, peptidoglycans, lipoteichoic acids, phosphatidyl cholines, bacteria-specific proteins, including lipoproteins, bacterial DNAs, viral single and double-stranded RNAs, unmethylated CpG-DNAs, mannans and a variety of other bacterial and fungal cell wall components. Such molecular patterns can also occur in other molecules such as plant alkaloids. These targets of innate immune recognition are called Pathogen Associated Molecular Patterns (PAMPs) since they are produced by microorganisms and not by the infected host organism. (Janeway et al., Ann. Rev. Immunol. 2002, 20:197-216; Medzhitov et al., Curr. Opin. Immunol. 1997, 94: 4-9).

Recent studies have demonstrated that the innate immune system plays a crucial role in the control of initiation of the adaptive immune response and in the induction of appropriate cell effector responses (Fearon et al., Science 1996, 272:50-3; Medzhitov et al., Cell 1997, 91:295-8). Initiation of effective immune responses requires the activation of the innate immune system by binding of a PAMP to its cognate Pattern Recognition Receptor (PRR) expressed on antigen-presenting cells (Medzhitov et al, op cit.; Barton et al., Current Opinion Immunol. 2002, 14:380-383.). The best characterized innate immune receptors are members of the Toll-like family of molecules (Toll-like receptors, or TLRs). These receptors belong to the family of “Toll-like receptors” because they are homologous to the Drosophila Toll protein, which is involved both in dorsoventral patterning in Drosophila embryos and in the immune response in adult flies (Lemaitre et al., Cell 1996, 86:973-83). TLRs participate in recognition of structures such as bacterial cell wall components (e.g., lipoproteins and lipopolysaccharides), bacterial DNA sequences that contain unmethylated CpG residues, and bacterial flagellin (Schwandner et al., J. Biol. Chem. 1999, 274:174069; Yoshimura et al., J. Immunol. 1999, 163:1-5; Aliprantis et al., Science 1999, 285:736-9; reviewed in Janeway and Medzhitov, op cit.). The binding of PAMPs to TLRs activates well-characterized immune pathways that can be mobilized for the development of more potent vaccines.

It has recently been discovered that a vaccine design should ensure that every antigen-presenting cell (APC) that is exposed to pathogen-derived antigen also receives an innate immune signal, and vice versa. This can be effectively achieved by designing the vaccine to contain an antigen-PAMP fusion construct, e.g., a contiguous fusion protein or conjugate consisting of PAMP:antigen(s). Such molecules would be expected to trigger signal transduction pathways in their target cells that result in the display of co-stimulatory molecules on the cell surface, as well as antigenic peptide in the context of major histocompatability context molecules.

The concept of incorporating triggers of the innate immune response into antigen-specific vaccines has been validated in the laboratory of Dr. Ruslan Medzhitov (Medzhitov et al., C. A., Cold Spring Harbor Symposia on Quantitative Biology 1999, LXIV:429-435. Innate immune induction of the adaptive immune response.) Immunization with recombinant PAMP-containing fusion proteins have previously been demonstrated to 1) induce antigen-specific T-cell and B-cell responses comparable to those induced by the use of conventional adjuvant; 2) result in significantly reduced non-specific inflammation; and 3) results in CD8 T cell-mediated protection that is specific for the fused antigen epitopes.

Diverse innate immune system receptors enable recognition of a wide range of pathogens and control the appropriate type of antigen-specific response that is triggered. Depending upon the cell type exposed to a PAMP and the PRR that binds to that PAMP, the profile of cytokines produced and secreted can influence whether the resultant adaptive immune response will be predominantly T-cell- or B-cell-mediated as well as the degree of inflammation accompanying the response. Since most TLRs signal through common intracellular pathways (NF-κB, Jun N-terminal kinase, mitogen-activated protein kinase), it is likely that some biological responses will be globally induced by any TLR signaling event. However, an emerging body of evidence demonstrates that divergent responses are induced by different TLRs (Hirschfeld et al., Infect. Immun. 2001, 69:1477-1482; Re et al., J. Biol. Chem. 2001, 276:37692-37699; Pulendran et al., J. Immunol. 2001, 167:5067-5076).

Thus, there exists a need for more and improved methods of enhancing the immune response against L. monocytogenes.

### SUMMARY OF THE INVENTION

The present invention relates to compositions comprising a pathogen associated molecular pattern (PAMP) that activate the TLR2 signaling or the TLR5 signaling of the innate host immune response and an antigenic polypeptide of Listeria monocytogenes. The composition can be a fusion protein, which may comprise a recombinant fusion protein. Antigen linked stimulation of specific TLR signaling pathways will lead to an targeted immune response to Listeria monocytogenes, an advantage over previously utilized nonspecific methods of stimulating an immune response.

In one embodiment, the invention is a composition comprising pathogen associated molecular pattern that activates at least one member selected from the group consisting of TLR2 and TLR5 and at least two distinct Listeria monocytogenes antigens.

In another embodiment, the invention is a composition comprising SEQ ID NO: 12.

In still another embodiment, the invention is a composition comprising SEQ ID NO: 16.

In an additional embodiment, the invention is a composition encoded by nucleic acid sequence comprising SEQ ID NO: 11.

In still another embodiment, the invention is a composition comprising SEQ ID NO: 14.

In a further embodiment, the invention is a composition encoded by nucleic acid sequence comprising SEQ ID NO: 13.

In still another embodiment, the invention is a composition comprising a pathogen associated molecular pattern that activates at least one member selected from the group consisting of TLR2 and TLR5 and a Listeria monocytogenes antigen that is not listeriolysin.

In another embodiment, the invention is a composition comprising a pathogen associated molecular pattern that activates at least one member selected from the group consisting of TLR2 and TLR5 and a Listeria monocytogenes p60 antigen.

In a further embodiment, the invention is a nucleic acid construct encoding a pathogen associated molecular pattern that activates at least one member selected from the group consisting of TLR2 and TLR5 and at least two distinct Listeria monocytogenes antigens.

In another embodiment, the invention is a nucleic acid construct encoding SEQ ID NO: 12.

In yet another embodiment, the invention is a nucleic acid construct encoding SEQ ID NO: 16.

In still another embodiment, the invention is a nucleic acid construct comprising SEQ ID NO: 11.

In another embodiment, the invention is a nucleic acid construct encoding SEQ ID NO: 14.

In yet another embodiment, the invention is a nucleic acid construct comprising SEQ ID NO:13.

In an additional embodiment, the invention is a nucleic acid construct encoding a pathogen associated molecular pattern that activates at least one member selected from the group consisting of TLR2 and TLR5 and a Listeria monocytogenes antigen that is not listeriolysin.

In a further embodiment, the invention is a nucleic acid construct encoding a pathogen associated molecular pattern that activates at least one member selected from the group consisting of TLR2 and TLR5; and a Listeria monocytogenes p60 antigen.

In a further embodiment, the invention is a method of producing a fusion protein, comprising the steps of culturing a host cell comprising a vector, wherein the vector comprises a nucleic acid construct encoding a fusion protein. The nucleic acid construct encodes a fusion protein that includes a pathogen associated molecular pattern that activates at least one member selected from the group consisting of TLR2 and TLR5; and at least two distinct Listeria monocytogenes antigens. The fusion protein produced by the host cell is isolated.

In another embodiment, the invention is a method of producing a fusion protein, comprising the steps of culturing the host cell comprising a vector, when the vector comprises a nucleic acid construct encoding a fusion protein. The nucleic acid construct encoding the fusion protein includes a pathogen associated molecular pattern that activates at least one member selected from the group consisting of TLR2 and TLR5 and a Listeria monocytogenes antigen that is not listeriolysin. The fusion protein produced by the host cell is isolated.

In another embodiment, the invention is a method of producing a fusion protein, comprising the steps of culturing a host cell comprising a vector, wherein the vector comprises a nucleic acid construct encoding a fusion protein. The nucleic acid construct encoding the fusion protein includes a pathogen associated molecular pattern that activates at least one member selected from the group consisting of TLR2 and TLR5; and Listeria monocytogenes p60 antigen. The fusion protein produced by the host cell is isolated.

In yet another embodiment, the invention is a method of stimulating the immune system in a subject, comprising the step of administering to the subject a composition including a pathogen associated molecular pattern that activates at least one member selected from the group consisting of TLR2 and TLR5 and at least two distinct Listeria monocytogenes antigens.

In another embodiment, the invention is a method of stimulating the immune system in a subject, comprising the step of administering to the subject a composition including a pathogen associated molecular pattern that activates at least one member selected from the group consisting of TLR2 and TLR5 and a Listeria monocytogenes antigen that is not listeriolysin. group consisting of TLR2 and TLR5 and a Listeria monocytogenes antigen that is not listeriolysin.

In yet another embodiment, the invention is a method of stimulating the immune system in a subject, comprising the step of administering to the subject a composition including a pathogen associated molecular pattern that activates at least one member selected from the group consisting of TLR2 and TLR5 and a Listeria monocytogenes p60 antigen.

In another embodiment, the invention is a PAMP that activates TLR2, such as E. coli bacterial lipoprotein or E. coli outer membrane protein A. In yet another embodiment, the PAMP which activates TLR5 is S. typhimurium fljB. In still another embodiment, the amino acid sequence of the Listeria antigen comprises at least one of SEQ ID NO: 7 (Listeria p60) and/or SEQ ID NO: 8 (Listeria LLO).

In yet another embodiment, the composition comprises a PAMP that activates TLR2 signaling comprising SEQ ID NO: 1 and an antigen comprises SEQ ID NO: 7 and SEQ ID NO: 8. In another embodiment, the composition comprises a PAMP which activates TLR2 signaling comprising SEQ ID NO: 3 and an antigen comprising SEQ ID NO: 7 and SEQ ID NO: 8. In another embodiment of the composition a PAMP that activates TLR5 signaling comprising SEQ ID NO: 5 and an antigen comprising SEQ ID NO: 7 and SEQ ID NO: 8.

In another embodiment, the invention is a method of inducing T-cell and/or B-cell responses, which are not dependent on CD4-mediated T help, in a subject by administering a composition of the invention (e.g., P2.LIST). The subject can be a rodent (e.g., a mouse, a rat), or a primate (e.g., a monkey, a human). The method of stimulating T-cell and/or B-cell responses can include production of at least one member selected from the group consisting of IgG1, IgG2a and IgG2b. The subject can have a condition associated with suboptimal, impaired, defective or absent CD4 T helper cell responses, as in, for example, HIV and Hepatitis C infection.

The invention is also directed to nucleic acid constructs, vectors, host cells, and methods for producing a fusion protein encoding a pathogen associated molecular pattern that activates TLR2 signaling or TLR5 signaling of the innate host immune response and an antigen of Listeria monocytogenes. Furthermore, the present invention provides compositions comprising PAMPs and Listeria monocytogenes antigens for use in the prevention of Listeria infection and protection against Listeria infection.

### BRIEF DESCRIPTION OF THE DRAWINGS

FIG. 1 depicts the amino acid sequence of the P2.LIST construct (SEQ ID NO.: 42) highlighting the P2.LLO.p60 fusion and displaying most of the known MHC class I and II epitopes. P2 residues are shaded, LLO (listeriolysin) residues are shown in bold, and p60 residues are shown in italics. CD4 and CD8 T cell epitopes are underscored.

FIG. 2 depicts plasmid constructs in pCRT7/CT-TOPO expression vector. Diagram is not drawn to scale. ST fljB: Salmonella typhimurium fljB full-length; LLO-p60: LLO₄₈₋₃₇₉ fused to p60₁₉₃₋₃₁₉; P2: E. coli truncated BLP1-24.

FIG. 3 depicts protection derived from wild type Listeria challenge following immunization with STF2.LIST.

FIGS. 4A, 4B and 4C depict STF2.OVA elicitation of TLR5 specific activity in vitro. T293 cells are TLR5 positive, 3T3 cells are TLR5 negative and RAW cells are TLR5 negative.

FIGS. 5A and 5B depict induction of OVA-specific antibody responses following immunization with STF2.OVA. Mice were immunized with equivalent doses of STF2.OVA fusion protein or CFA/OVA. Seven days later, the mice were bled and sera were collected (solid bars), or mice were challenged by oral administration of LM.OVA and bled five days following challenge (stippled bars). OVA-specific IgG₁ and IgG₂ₐ were measured in the sera by ELISA. Results depict mean±standard deviation from 4 mice per group.

FIGS. 6A and 6B depict evaluation of antigen specific CD8 T cell responses by IFNγ ELISPOT. Lymphocytes from the draining lymph nodes of mice described in FIG. 5 were restimulated overnight with SIINFEKL peptide. IFNγ-secreting cells were detected using commercial ELISPOT reagents. Results depict mean±standard error from 3 animals per group.

FIGS. 7A and 7B depict TLR5-specific activity of the STF2.LIST fusion protein in vitro. Stimulation of the TLR4⁻5⁺ HEK293 cells with STF2.LIST (▪) results in NF-κB mediated luciferase activity similar to that observed following stimulation with recombinant flagellin (♦) (top). LPS() activates the TLR4⁺5⁻ RAW cell line with little to no activation observed following stimulation with STF2.LIST, in the presence (▪) or absence (□) of polymyxin B (Lower). These data demonstrate the TLR5 specificity and absence of possible endotoxin in the purified STF2.LIST fusion protein.

FIGS. 8A and 8B depict immunization with STF2.LIST that induces antigen-specific CD8 T cell responses and protective immunity. Mice received a single s.c. immunization with PBS, STF2 (12 μg) or STF2.LIST (25 μg). FIG. 8A-One week later, spleen cells were harvested and restimulated in vitro for 24 hours with the H-2K^{d} restricted immunodominant epitope LLO₉₁₋₉₉, and IFNγ ELISPOT responses were measured. FIG. 8B—An identical cohort (n=10/group) were challenged with 2×10³ CFU of wild type L. monocytogenes, and bacterial burden in the spleen was measured three days post-challenge. P values by Students t-test: PBS vs. STF2=0.12; PBS vs. STF2.LIST=0.06.

FIG. 9 depicts the amino acid (aa) sequence of the signal sequence and lipidation domain of E. coli lipoprotein (BLP) (SEQ ID NO.: 1), GenBank Accession No. X68953. A truncated portion of BLP for use in the compositions and fusion proteins of the invention is the lipidation domain of BLP, amino acids 21-24 (CSSN) of SEQ ID NO.: 1.

FIG. 10 depicts the nucleic acid (na) sequence for the BLP signal sequence and lipidation domain (SEQ ID NO.: 2).

FIG. 11 depicts E. coli outer membrane protein A (OmpA) (SEQ ID NO.: 3), GenBank Accession No. AAC74043.1 and AE000198.1.

FIG. 12 depicts the nucleic acid sequence of E. coli (OmpA) (SEQ ID NO.: 4):

FIG. 13 depicts the amino acid sequence of STF2 emb (SEQ ID NO.: 5) of Salmonella typhimurium fljB flagellin, GenBank Accession No. AF045151.

FIG. 14 depicts the nucleic acid sequence of STF2 specific emb (SEQ ID NO.: 6).

FIG. 15 depicts the amino acid sequence of Listeria monocytogenes p60 (SEQ ID NO.: 7).

FIG. 16 depicts the amino acid sequence of Listeria monocytogenes LLO (SEQ ID NO.: 8).

FIG. 17 depicts the nucleic acid sequence of Listeria monocytogenes p60 (SEQ ID NO.: 9).

FIG. 18 depicts the nucleic acid sequence of Listeria monocytogenes LLO (SEQ ID NO.: 10).

FIG. 19 depicts the nucleic acid sequence of P2. LIST (P2-LLO-p60) (SEQ ID NO.: 11).

FIG. 20 depicts the amino acid sequence of P2. LIST (P2-LLO-p60) (SEQ ID NO.: 12)

FIG. 21 depicts the nucleic acid sequence of STF2. LIST (STF2-LLO-p60) (SEQ ID NO.: 13).

FIG. 22 depicts the amino acid sequence of STF2. LIST (STF2-LLO-p60) (SEQ ID NO.: 14).

FIG. 23 depicts the nucleic acid sequence of P2.LIST (P2-LLO-p60) after cleavage of the signal sequence in bacteria (SEQ ID NO.: 15).

FIG. 24 depicts the amino acid sequence of P2.LIST (P2-LLO-p60) after cleavage of the signal sequence in bacteria (SEQ ID NO.: 16).

FIG. 25 depicts a truncated Salmonella typhimurium fljB (STF2Δ) in which amino acids 176-404 of SEQ ID NO.: 5 have been deleted (SEQ ID NO.: 17).

FIG. 26 depicts the amino acid sequence of the truncated Salmonella typhimurium fljB (STF2Δ) (SEQ ID NO.: 18) containing a linker sequence GAPVDPASPW (SEQ ID NO.: 19), which is encoded by the nucleic acid sequence GGAGCGCCGGTGGATCCTGCTAGCCCATGG (SEQ ID NO.: 20).

FIG. 27 depicts the nucleic acid sequence of the truncated Salmonella typhimurium fljB (STF2Δ) (SEQ ID NO.: 21). The peptide linker of SEQ ID NO: 19 is indicated in underline (GGAGCGCCGGTGGATCCTGCTAGCCCATGG—SEQ ID NO: 49).

FIGS. 28A and 28B depict antigen-specific T and B cell responses following immunization with STF2.OVA: C57BL/6 mice were immunized on day 0 and 14 with the equivalent concentration (12 μg) of OVA mixed with STF2 (STF2+OVA) or the recombinant fusion protein STF2.OVA. Animals were sacrificed and evaluated for primary antigen-specific T cell (FIG. 28A) and secondary B cell (FIG. 28B) responses by ELISPOT and ELISA, respectively. For ELISPOT assays purified CD4 and CD8 T cells were stimulated for 24 hours with OVA or the MHC Class I-restricted OVA-derived peptide SIINFEKL, respectively. IgG-specific responses were determined by direct ELISA following detection with anti-mouse HRP-labeled IgG1, IgG2a or IgG2b specific secondary antibodies.

FIGS. 29A and 29B depict STF2.OVA mediated protection from bacterial challenge in vivo. C57BL/6 mice were immunized s.c. with OVA (12 μg, hatched bars) or STF2.OVA (25 μg, solid bars) or PBS. FIG. 29A: Nine days later, CD8 T cells were isolated and restimulated in vitro with the OVA-derived MHC Class I restricted peptides SIINFEKL in IFNα ELISPOT assays. FIG. 29B: On day 21 post immunization, a second cohort of mice was challenged i.v. with 1×10⁴ CFU of recombinant OVA-expressing L. monocytogenes. Bacterial burden in the spleen was measured three days post-challenge. Data depict the mean±SD of 10 mice per group. The data are representative of results from three experiments with similar results (*P<0.05 by paired Student's t-Test).

FIGS. 30A, 30B and 30C depict immunization with STF2.LIST induces antigen-specific CD8 T cell responses and protective immunity. FIG. 30A: BALB/c mice were immunized s.c. with PBS, STF2.LIST (25 μg), or 2×10³ CFU of L. monocytogenes (L.m.) as positive controls. Antigen-specific CD8 T cells responses to the H-2K^{d} restricted immunodominant epitopes LLO91-99 (FIG. 30A) and p60₂₁₇₋₂₂₅ (FIG. 30B) were evaluated in IFNγ ELISPOT assays on day 9 post immunization (□) and 3 days following a challenge with 2×10⁴ CFU of L. monocytogenes on day 21 (▪). FIG. 30C: Antigen-specific protection in vivo was examined in BALB/c mice following immunization with PBS, STF2 (12 μg) or STF2.LIST (25 μg). On day 21 post immunization animals were challenged i.v. with 2×10⁴ CFU of L. monocytogenes. Bacterial burden in the spleen was measured three days post-challenge. Data depict the mean±SD of 10 mice per group. The data are representative of results from three experiments with similar results (*P<0.05 by paired Student's t-test).

FIG. 31 depicts the nucleic acid sequence encoding the STF2.OVA fusion protein (SEQ ID NO: 50). The portion of the nucleic acid encoding OVA is indicated by bolded letters.

FIG. 32 depicts the amino acid sequence (SEQ ID NO: 51) encoded by SEQ ID NO: 50. The amino acid sequence of OVA is indicated by bolded letters.

### DETAILED DESCRIPTION OF THE INVENTION

The features and other details of the invention, either as steps of the invention or as combinations of parts of the invention, will now be more particularly described and pointed out in the claims. It will be understood that the particular embodiments of the invention are shown by way of illustration and not as limitations of the invention. The principle features of this invention can be employed in various embodiments without departing from the scope of the invention.

In one embodiment, the invention is a composition comprising a pathogen associated molecular pattern that activates at least one member selected from the group consisting of TLR2 and TLR5; and at least two distinct Listeria monocytogenes antigens.

The pathogen associated molecular pattern and Listeria monocytogenes antigens can be components of a fusion protein. The pathogen associated molecular pattern can be fused or linked to the amino or carboxy terminus of one of the Listeria monocytogenes antigens. The fusion protein can further include a linker between the pathogen associated molecular antigen and the Listeria monocytogenes antigen The linker can be a peptide linker, such as SEQ ID NO: 19, encoded by SEQ ID NO: 20. The peptide linker can be at least one member selected from the group consisting of a lysine residue, a glutamic acid residue, a glycine residue, a serine residue and an arginine residue. The linker can be between at least two pathogen associated molecular antigens. The linker can also be between at least two antigens of Listeria monocytogenes.

The pathogen associated molecular pattern can activate a TLR2 signaling pathway. The pathogen associated molecular pattern can be at least one member selected from the group consisting of BLP, such as E. coli BLP and OmpA. The E. coli BLP can include at least a fragment of SEQ ID NO: 1, such as the lipidation domain of BLP (e.g., amino acid sequence cysteine-serine-serine-asparagine (CSSN)).

The OmpA can include E. coli OmpA, such as at least a fragment of SEQ ID NO: 3.

The pathogen associated molecular pattern can include at least a fragment of SEQ ID NO: 1 and the Lysteria monocytogenes antigens include at least a fragment of each of SEQ ID NO: 7 and SEQ ID NO: 8.

The pathogen associated molecular pattern can activate a TLR5 signaling pathway. The pathogen associated molecular pattern can be at least a fragment of a flagellin. The flagellin can include a polypeptide selected from the group consisting of H. pylori, V. cholera, S. marcesens, S. flexneri, T. pallidum, L. pneumophilia; B burgdorferei; C. difficile, R. meliloti, A. tumefaciens; R. lupine; B. clarridgeiae, P. mirabilis, B. subtilus, L. monocytogenes, P. aeruginosa and E. coli.

In a particular embodiment, the flagellin is selected from the group consisting of S. typhimurium fljB and E. coli FliC. The S. typhimurium fljB can include at least a fragment of SEQ ID NO: 5, such as SEQ ID NO: 18, encoded by SEQ ID NO: 21.

The pathogen associated molecular pattern can be a fragment selected from at least one member of the group consisting of the Oprl protein of P. aeruginosa; the fibril subunit protein of Porphyromonas gingivalis; the maerophagic activating lipopeptide 2 (MALP-2) of Mycoplasma fermentans; and p19 from Mycobacterium tuberculosis.

The pathogen associated molecular pattern can also be at least one member selected from the group consisting of lipopolysaccharides; phosphatidyl choline; glucans; peptidoglycans; teichoic acids; lipoteichoic acids; proteins; lipoproteins; lipopeptides; outer membrane proteins (OMPs), outer surface proteins (OSPs); protein components of bacterial cell walls; flagellins; bacterial DNAs; single and double-stranded viral RNAs; unmethylated CpG-DNAs; mannans; mycobacterial membranes; and porins.

The Listeria monocytogenes antigens can include at least a fragment of each of SEQ ID NO: 7 and SEQ ID NO: 8.

The Listeria monocytogenes antigens can be encoded by the nucleic acid sequences that includes a fragment (also referred to herein as a subsequence) of at least one of SEQ ID NO: 9 and SEQ ID NO: 10.

The pathogen associated molecular pattern can include at least a fragment of SEQ ID NO: 3 and the Listeria monocytogenes antigens can include at least a fragment of each of SEQ ID NO: 7 and SEQ ID NO: 8. The pathogen associated molecular pattern can include at least a fragment of SEQ ID NO: 5 and the Listeria monocytogenes antigens can include at least a fragment of each of SEQ ID NO: 7 and SEQ ID NO: 8. The pathogen associated molecular pattern can also include at least a fragment of SEQ ID NO: 1 and the Listeria monocytogenes antigens include at least a fragment of each of SEQ ID NO: 7 and SEQ ID NO: 8. The composition can comprise at least one member selected from the group consisting of SEQ ID NO: 12, SEQ ID NO: 16, a nucleic acid sequence comprising SEQ ID NO: 11; SEQ ID NO: 14; a nucleic acid sequence comprising SEQ ID NO: 13.

In still another embodiment, the invention is a composition comprising a pathogen associated molecular pattern that activates at least one member selected from the group consisting of TLR2 and TLR5 and a Listeria monocytogenes antigen that is not listeriolysin.

In another embodiment, the invention is a composition comprising a pathogen associated molecular pattern that activates at least one member selected from the group consisting of TLR2 and TLR5 and a Listeria monocytogenes p60 antigen, which can further include at least one additional Listeria monocytogenes antigen (e.g., listeriolysin).

In a further embodiment, the invention is a nucleic acid construct encoding a pathogen associated molecular pattern that activates at least one member selected from the group consisting of TLR2 and TLR5 and at least two distinct Listeria monocytogenes antigens. The nucleic acid construct can be components of a fusion protein. The nucleic acid construct can be at least one member selected from the group consisting of SEQ ID NO: 11 and 13. The nucleic acid construct can be at least one member selected from the group consisting a nucleic acid sequence encoding SEQ ID NO: 12, SEQ ID NO: 16 and SEQ ID NO: 14.

In an additional embodiment, the invention is a nucleic acid construct encoding a pathogen associated molecular pattern that activates at least one member selected from the group consisting of TLR2 and TLR5 and a Listeria monocytogenes antigen that is not listeriolysin.

In a further embodiment, the invention is a nucleic acid construct encoding a pathogen associated molecular pattern that activates at least one member selected from the group consisting of TLR2 and TLR5 and Listeria monocytogenes p60 antigen, which can further include at least one additional Listeria monocytogenes antigen, such as listeriolysin.

In yet another embodiment, the invention is a vector comprising the nucleic acid constructs of the invention and host cells (prokaryote and eukaroyte) comprising the vectors of the invention.

In a further embodiment, the invention is a method of producing a fusion protein, comprising the steps of culturing a host cell comprising a vector, wherein the vector comprises a nucleic acid construct encoding a fusion protein. The nucleic acid construct encodes a fusion protein that includes a pathogen associated molecular pattern that activates at least one member selected from the group consisting of TLR2 and TLR5; and at least two distinct Listeria monocytogenes antigens. The fusion protein produced by the host cell is isolated.

In another embodiment, the invention is a method of producing a fusion protein, comprising the steps of culturing the host cell comprising a vector, when the vector comprises a nucleic acid construct encoding a fusion protein. The nucleic acid construct encoding the fusion protein includes a pathogen associated molecular pattern that activates at least one member selected from the group consisting of TLR2 and TLR5 and a Listeria monocytogenes antigen that is not listeriolysin. The fusion protein produced by the host cell is isolated. Techniques to isolate the fusion protein from the host cell are well known to one of skill in the art and can include cell lysis, chromatography and cell separation techniques.

In another embodiment, the invention is a method of producing a fusion protein, comprising the steps of culturing a host cell comprising a vector, wherein the vector comprises a nucleic acid construct encoding a fusion protein. The nucleic acid construct encoding the fusion protein includes a pathogen associated molecular pattern that activates at least one member selected from the group consisting of TLR2 and TLR5 and Listeria monocytogenes p60 antigen. The fusion protein produced by the host cell is isolated.

In yet another embodiment, the invention is a method of stimulating the immune system in a subject, comprising the step of administering to the subject a composition including a pathogen associated molecular pattern that activates at least one member selected from the group consisting of TLR2 and TLR5 and at least two distinct Listeria monocytogenes antigens.

In an additional embodiment, the invention is a method of stimulating the immune system in a subject, comprising the step of administering to the subject a composition including a pathogen associated molecular pattern that activates at least one member selected from the group consisting of TLR2 and TLR5 and a Listeria monocytogenes antigen that is not listeriolysin.

In yet another embodiment, the invention is a method of stimulating the immune system in a subject, comprising the step of administering to the subject a composition including a pathogen associated molecular pattern that activates at least one member selected from the group consisting of TLR2 and TLR5 and Listeria monocytogenes p60 antigen.

The invention provides a composition (e.g., fusion protein vaccine construct) comprising a pathogen associated molecular pattern (PAMP) protein that activates toll-like receptor 2 (TLR2) or toll-like receptor 5 (TLR5) signaling and an antigen from Listeria monocytogenes. PAMPs employed in the compositions include E. coli bacterial lipoprotein signal sequence and lipidation motif conjugated with Listeria antigenic poloypeptides, E. coli outer membrane protein conjugated with Listeria antigenic polypeptides, and flagellin conjugated with Listeria antigenic polypeptides, which activate TLR2, and TLR5 signaling, respectively. Amino acids, nucleotides, vectors, cell lines, and the methods for production and use of the vaccine construct as a vaccine to produce an immune response are also provided. Linking TLR activation with antigen presentation results in a safer, more potent, protective antigen-specific immune response compared to immunization with the same antigen(s) in traditional adjuvants.

An embodiment of the invention is recombinant DNA vectors encoding fusion proteins consisting of antigens derived from Listeria monocytogenes fused to polypeptide PAMPs that trigger TLR signaling. For example, expression and purification of biologically-active recombinant fusion proteins containing PAMPs and L. monocytogenes antigens, comparison of the efficacy of BLP- and flagellin-containing recombinant proteins in murine models of listeriosis; identification of the mechanisms of protection by examining cellular and humoral immune responses triggered by BLP- and flagellin-containing recombinant proteins. The mouse model of listeriosis (Geginat et al., J. Immunol. 2001, 166: 1877-1884) has provided insights into the mechanisms of disease and the immunological response to infection with L. monocytogenes. This model allows study of both short-term and memory responses, and to understand the cellular and humoral compartments responsible for protection. These studies lead to the identification of a PAMP that generates a robust protective immune response in mice.

Fusing L. monocytogenes antigens to bacterial lipoprotein (BLP) and flagellin separately permits comparison of the immune responses induced by the antigens in the context of signaling through different TLRs. In vitro assays demonstrate the contributions of the humoral and cell-mediated immune responses to each TLR challenge. Data from these experiments provides guidance as to the selection of appropriate PAMPs that will generate the phenotype of immune response best suited to protect against L. monocytogenes, the infectious organism.

“Pathogen-Associated Molecular Pattern” or “PAMP” refers to a molecular pattern found in a microorganism but not in humans, which, when it binds a PRR, can trigger an innate immune response. Thus, as used herein, the term “PAMP” includes any such microbial molecular pattern and is not limited to those associated with pathogenic microorganisms or microbes. As used herein, the term “PAMP” includes a PAMP, derivative or portion of a PAMP that is immunostimulatory, and any immunostimulatory molecule derived from any PAMP. These structures, or derivatives thereof, are potential initiators of innate immune responses, and therefore, ligands for PRRs, including Toll receptors and TLRs. PAMPs are found in, or composed of molecules including, but not limited to, lipopolysaccharides; phosphatidyl choline; glycans, including peptidoglycans; teichoic acids, including lipoteichoic acids; proteins, including lipoproteins and lipopeptides; outer membrane proteins (OMPs), outer surface proteins (OSPs) and other protein components of the bacterial cell walls and Flagellins; bacterial DNAs; single and double-stranded viral RNAs; unmethylated CpG-DNAs; mannans; mycobacterial membranes; porins; and a variety of other bacterial and fungal cell wall components, including those found in yeast. This invention uses PAMPs that bind to and activate TLR2 or TLR5 to prepare the vaccine constructs.

The term PAMP/antigen or PAMP:antigen refers to a molecular construct in which an antigen and a PAMP or PAMP mimetic are covalently or noncovalently linked. Any PAMP molecule can be associated with the antigen using chemical conjugation techniques (including peptide condensation), or using genetic engineering (i.e., recombinant technology), such as in the preparation of a fusion protein construct exemplified infra. A “PAMP/antigen fusion” or “PAMP/antigen chimera” refers to any protein fusion formed between a PAMP or PAMP mimetic and an antigen, whether by peptide condensation chemistry or recombinant expression technology.

The term “vaccine construct” means PAMP/antigen construct.

“Fusion protein” refers to any protein fusion comprising two or more domains selected from the following group consisting of: proteins, peptides, lipoproteins, lipopeptides, glycoproteins, glycopeptides, mucoproteins, mucopeptides, such that one domain is from a PAMP and the other domain is from a protein antigen. The term “fusion protein” also refers to an antigen or an immunogenic portion or derivative thereof which has been modified to contain an amino acid sequence that results in post-translational modification of that amino acid sequence or a portion of that sequence, wherein the post-translationally modified sequence is a ligand for a PRR. The amino acid sequence that results in post-translational modification to form a ligand for a PRR can comprise a consensus sequence, or that amino acid sequence can contain a leader sequence and a consensus sequence.

As used herein, the term “peptide” is intended to mean two or more amino acids covalently bonded together.

“Domain” refers to a portion of a protein with identifiable structure and/or function. The combination of domains in a protein determines its overall (tertiary) structure and, usually, function. An “antigen domain” comprises an antigen or an immunogenic portion or derivative of an antigen. A “PAMP domain” comprises a PAMP or a PAMP mimetic or an immunostimulatory portion or derivative of a PAMP or a PAMP mimetic.

An “antigenic polypeptide of Listeria monocyotgenes” (or simply, for purposes of this invention, an “antigenic polypeptide”) refers to a polypeptide from L. monocytogenes that contains epitopes recognized by T cells and B cells. These epitopes are also present on wildtype L. monocytogenes, and immune cells that are specific for these epitopes are able to mediate an adaptive immune response to the wildtype microorganism.

An “antigen,” as used herein, means any naturally occurring or synthetic molecule, such as a protein, peptide, lipid, carbohydrate, that generates an immune response, including, for example, a fragment of a naturally-occurring molecule, wherein the fragment generates an immune response. “Distinct antigens,” such as “distinct Listeria monocytogenes antigens,” refers to antigens that elicit immune responses to different epitopes of Listeria monocytogenes. A fragment, as used herein, in reference to an antigen, means a portion of the antigen that is isolated, separate or apart from the entire antigen. For example, a fragment of an antigen can be a portion of a protein antigen.

The term “recombinant” refers to genetic material that is produced by engineering genes, gene derivatives, or other genetic material. As used herein, “recombinant” also refers to the products produced from recombinant genes (e.g., recombinant protein).

A fragment of an amino acid or nucleic acid sequence (also referred to herein as a truncated amino acid or truncated nucleic acid sequence) refers to any portion or part of a sequence that generates an immune response (innate or adaptive immunity). For example, a fragment of SEQ ID NO: 1 is amino acid residues 21-24 CSSN of SEQ ID NO: 1; and a fragment of SEQ ID NO: 5 is SEQ ID NO: 17 or SEQ ID NO: 18, a truncated Salmonella typhimurium fljB, which has amino acid residues 1-169 and 416-506 of SEQ ID NO: 5 removed.

“Toll-like receptor” (TLR) refers to any of a family of receptor proteins that are homologous to the Drosophila melanogaster Toll protein. TLRs also refer to type I transmembrane signaling receptor proteins that are characterized by an extracellular leucine-rich repeat domain and an intracellular domain homologous to that of the interleukin 1 receptor. The TLR family includes, but is not limited to, mouse TLR2 and TLR5 and their homologues, particularly in other species including humans. This invention also defines Toll receptor proteins and TLRs wherein the nucleic acids encoding such proteins have at least about 70% sequence identity, more preferably, at least about 80% sequence identity, even more preferably, at least about 85% sequence identity, yet more preferably at least about 90% sequence identity, and most preferably at least about 95% sequence identity to the nucleic acid sequence encoding the Toll protein and the TLR proteins TLR2, TLR4 and TLR5 and other members of the TLR family.

A “TLR signaling pathway” is the intracellular signal transduction pathway employed by a particular TLR when activated by its cognate TLR ligand. Most TLRs signal through common intracellular pathways (NF-κB, Jun N-terminal kinase, mitogen-activated protein kinase), but divergent responses are induced by different TLRs (Hirschfeld et al, op cit.; Re et al., op cit. Pulendran et al, op cit.). The present invention provides for both general and specific activation of TLR2 and TLR5 signaling pathways by the PAMP:antigen constructs of the invention.

For purposes of the present invention, a “protective immune response” is an immune response that limits or clears invading Listeria microbes in a subject. As demonstrated in the examples, such an immune response involves antigen specific cytotoxic T lymphocyte activation. More generally, the protective immune response involves adaptive immunity enhance with antigen-associated innate immunity. “Adaptive immunity” refers to the adaptive immune system, which involves two types of receptors generated by somatic mechanisms during the development of each individual organism. As used herein, the “adaptive immune system” refers to both cellular and humoral immunity. Immune recognition by the adaptive immune system is mediated by antigen receptors, i.e., membrane-associated immunoglobulin and T cell receptor (TCR).

“Innate immunity” refers to the innate immune system, which, unlike the “adaptive immune system”, uses a set of germline-encoded receptors for the recognition of conserved molecular patterns present in microorganisms.

“Immunostimulatory” refers to the ability of a molecule to activate either the adaptive immune system or the innate immune system. As used herein, “activation” of either immune system includes the production of constituents of humoral and/or cellular immune responses that are reactive against the immunostimulatory molecule.

The term “immunotherapy” refers to a treatment regimen based on activation of a pathogen-specific immune response. A vaccine can be one form of immunotherapy.

The term “vaccine” is used herein in a general sense to refer to any therapeutic or immunogenic or immunostimulatory composition that includes the features of the present invention. The term “vaccine” refers to a composition (protein or vector; the latter may also be loosely termed a “DNA vaccine”, although RNA vectors can be used as well) that can be used to elicit protective immunity in a recipient. It should be noted that to be effective, a vaccine of the invention can elicit immunity in a portion of the population, as some individuals may fail to mount a robust or protective immune response, or, in some cases, any immune response. This inability may stem from the individual's genetic background or because of an immunodeficiency condition (either acquired or congenital) or immunosuppression (e.g., treatment with immunosuppressive drugs to prevent organ rejection or suppress an autoimmune condition). Efficacy can be established in animal models.

The term “protect” is used herein to mean prevent or treat, or both, as appropriate, a Listeria infection in a subject. Thus, prophylactic administration of the vaccine can protect the recipient subject from Listeria infection, e.g., to prevent Listeriosis. Therapeutic administration of the vaccine or immunotherapy can protect the recipient from L. monocytogenes infection mediated pathogenesis.

The term “subject” as used herein refers to an animal, in particular, a human.

The term “vector for expression in humans” as used herein means that the vector at least includes a promoter that is effective in human cells, and preferably that the vector is safe and effective in humans. Such a vector will, for example, omit extraneous genes not involved in developing immunity. If it is a viral vector, it will omit regions that permit replication and development of a robust infection, and will be engineered to avoid development of replication competence in vivo. Such vectors are preferably safe for use in humans; in a more preferred embodiment, the vector is approved by a government regulatory agency (such as the Food and Drug Administration) for clinical testing or use in humans. Specific vectors are described in greater detail below.

When formulated in a pharmaceutical composition, a therapeutic compound can be admixed with a pharmaceutically acceptable carrier or excipient. The phrase “pharmaceutically acceptable” refers to molecular entities and compositions that are “generally regarded as safe”, e.g., that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and, more particularly, in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin. The compositions and fusion proteins of the invention can be administered orally, intravenously, intrapentoneally, subcutaneously or intramuscularly.

“Therapeutically effective amount” refers to an amount of an agent (e.g., a vaccine) that can produce a measurable positive effect in a patient. In the context of the present invention, this includes reducing bacterial burden in a subject.

A subject in whom administration of the vaccine compound is an effective therapeutic regiment for a disease or disorder is preferably a human, but can be any animal, including a laboratory animal in the context of a clinical trial or screening or activity experiment. Thus, as can be readily appreciated by one of ordinary skill in the art, the methods and compositions of the present invention are particularly suited to administration to any animal, particularly a mammal, and including, but by no means limited to, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for veterinary medical use.

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “isolated” means that the referenced material is removed from its native environment, e.g., a cell. Thus, an isolated biological material can be free of some or all cellular components, i.e., components of the cells in which the native material occurs naturally (e.g., cytoplasmic or membrane component). A material shall be deemed isolated if it is present in a cell extract or if it is present in a heterologous cell or cell extract. In the case of nucleic acid molecules, an isolated nucleic acid includes a PCR product, an isolated mRNA, a cDNA, or a restriction fragment. In another embodiment, an isolated nucleic acid is preferably excised from the chromosome in which it may be found, and more preferably is no longer joined or proximal to non-coding regions (but may be joined to its native regulatory regions or portions thereof), or to other genes, located upstream or downstream of the gene contained by the isolated nucleic acid molecule when found in the chromosome. In yet another embodiment, the isolated nucleic acid lacks one or more introns. Isolated nucleic acid molecules include sequences inserted into plasmids, cosmids, artificial chromosomes, and the like, i.e., when it forms part of a chimeric recombinant nucleic acid construct. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The term “purified” as used herein refers to material that has been isolated under conditions that reduce or eliminate the presence of unrelated materials, i.e., contaminants, including native materials from which the material is obtained. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. As used herein, the term “substantially free” is used operationally, in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art.

Methods for purification are well known in the art. For example, nucleic acids can be purified by precipitation, chromatography (including without limitation preparative solid phase chromatography, oligonucleotide hybridization, and triple helix chromatography), ultracentrifugation, and other means. Polypeptides and proteins can be purified by various methods including, without limitation, preparative disc gel electrophoresis and isoelectric focusing; affinity, HPLC, reversed phase HPLC, gel filtration or size exclusion, ion exchange and partition chromatography; precipitation and salting-out chromatography; extraction; and countercurrent distribution. For some purposes, it is preferable to produce the polypeptide in a recombinant system in which the protein contains an additional sequence tag that facilitates purification, such as, but not limited to, a polyhistidine sequence, or a sequence that specifically binds to an antibody, such as FLAG and GST. The polypeptide can then be purified from a crude lysate of the host cell by chromatography on an appropriate solid phase matrix. Alternatively, antibodies produced against the protein or against peptides derived there from can be used as purification reagents. Cells can be purified by various techniques, including centrifugation, matrix separation (e.g., nylon wool separation), panning and other immunoselection techniques, depletion (e.g., complement depletion of contaminating cells), and cell sorting (e.g., fluorescence activated cell sorting (FACS)). Other purification methods are possible and contemplated herein. A purified material may contain less than about 50%, preferably less than about 75%, and most preferably less than about 90%, of the cellular components, media, proteins, or other nondesirable components or impurities (as context requires), with which it was originally associated. The term “substantially pure” indicates the highest degree of purity which can be achieved using conventional purification techniques known in the art.

### PAMP: Antigen Constructs

In accordance with the present invention there may be employed conventional molecular biology, microbiology, and recombinant DNA techniques within the skill of the art. Such techniques are explained fully in the literature. See, e.g., Sambrook et al., Molecular Cloning: A Laboratory Manual, Second Edition 1989; Glover, ed., DNA Cloning: A Practical Approach, Volumes I and II 1989; Gait, ed., Oligonucleotide Synthesis 1984; Hames et al., ed., Nucleic Acid Hybridizationl985; Hames et al., ed., Transcription And Translation 1984; Freshney, ed., Animal Cell Culture 1986); Immobilized Cells And Enzymes, IRL Press 1986; B. Perbal, A Practical Guide To Molecular Cloning 1984; Ausubel et al., ed., Current Protocols in Molecular Biology 1994.

“Amplification” of DNA as used herein denotes the use of polymerase chain reaction (PCR) to increase the concentration of a particular DNA sequence within a mixture of DNA sequences. For a description of PCR see Saiki et al., Science 1988, 239:487.

A “polynucleotide” or “nucleotide sequence” is a series of nucleotide bases (also called “nucleotides”) in a nucleic acid, such as DNA and RNA, and means any chain of two or more nucleotides. A nucleotide sequence typically carries genetic information, including the information used by cellular machinery to make proteins and enzymes. These terms include double or single stranded genomic and cDNA, RNA, any synthetic and genetically manipulated polynucleotide, and both sense and anti-sense polynucleotide (although only sense stands are being represented herein). This includes single- and double-stranded molecules, i.e., DNA-DNA, DNA-RNA and RNA-RNA hybrids, as well as “protein nucleic acids” (PNA) formed by conjugating bases to an amino acid backbone. This also includes nucleic acids containing modified bases, for example thio-uracil, thio-guanine and fluoro-uracil.

The nucleic acids encoding the PAMP, antigen, or PAMP:antigen herein may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, biotin, and the like.

A “promoter” or “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease SI), as well as protein binding domains (consensus sequences) responsible for the binding of RNA polymerase. The promoter may be operatively associated with other expression control sequences, including enhancer and repressor sequences.

Promoters which may be used to control gene expression include, but are not limited to, cytomegalovirus (CMV) promoter (U.S. Pat. No. 5,385,839 and No. 5,168,062), the SV40 early promoter region (Benoist and Chambon, Nature 1981, 290:304 310), the promoter contained in the 3′ long terminal repeat of Rous sarcoma virus (Yamamoto et al., Cell 1980, 22:787 797), the herpes thymidine kinase promoter (Wagner et al., Proc. Natl. Acad. Sci. USA 1981, 78:1441 1445), the regulatory sequences of the metallothionein gene (Brinster et al., Nature 1982, 296:39-42); prokaryotic expression vectors such as the beta lactamase promoter (VIIIa Komaroff et al., Proc. Natl. Acad. Sci. USA, 1978, 75:3727 3731), or the tac promoter (DeBoer et al., Proc. Natl. Acad. Sci. USA, 1983, 80:21 25); see also “Useful proteins from recombinant bacteria” in Scientific American 1980, 242:74 94; promoter elements from yeast or other fungi such as the Gal 4 promoter, the ADC (alcohol dehydrogenase) promoter, PGK (phosphoglycerol kinase) promoter, alkaline phosphatase promoter.

A “coding sequence” or a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

A coding sequence is “under the control of” or “operatively associated with” transcriptional and translational control sequences in a cell when RNA polymerase transcribes the coding sequence into RNA, particularly mRNA, which is then trans-RNA spliced (if it contains introns) and translated into the protein encoded by the coding sequence.

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. Vectors include plasmids, phages, viruses, etc.; they are discussed in greater detail below.

Vectors typically comprise the DNA of a transmissible agent, into which foreign DNA is inserted. A common way to insert one segment of DNA into another segment of DNA involves the use of enzymes called restriction enzymes that cleave DNA at specific sites (specific groups of nucleotides) called restriction sites. A “cassette” refers to a DNA coding sequence or segment of DNA that codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.” A common type of vector is a “plasmid”, which generally is a self-contained molecule of double-stranded DNA, usually of bacterial origin, that can readily accept additional (foreign) DNA and which can readily introduced into a suitable host cell. A plasmid vector often contains coding DNA and promoter DNA and has one or more restriction sites suitable for inserting foreign DNA. Coding DNA is a DNA sequence that encodes a particular amino acid sequence for a particular protein or enzyme. Promoter DNA is a DNA sequence which initiates, regulates, or otherwise mediates or controls the expression of the coding DNA. Promoter DNA and coding DNA may be from the same gene or from different genes, and may be from the same or different organisms. A large number of vectors, including plasmid and fungal vectors, have been described for replication and/or expression in a variety of eukaryotic and prokaryotic hosts. Non-limiting examples include pKK plasmids (Clonetech), pUC plasmids, pET plasmids (Novagen, Inc., Madison, Wis.), pRSET or pREP plasmids (Invitrogen, San Diego, Calif.), or pMAL plasmids (New England Biolabs, Beverly, Mass.), and many appropriate host cells, using methods disclosed or cited herein or otherwise known to those skilled in the relevant art. Recombinant cloning vectors will often include one or more replication systems for cloning or expression, one or more markers for selection in the host, e.g. antibiotic resistance, and one or more expression cassettes.

The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a protein. The expression product itself, e.g. the resulting protein, may also be said to be “expressed” by the cell. An expression product can be characterized as intracellular, extracellular or secreted. The term “intracellular” means something that is inside a cell. The term “extracellular” means something that is outside a cell. A substance is “secreted” by a cell if it appears in significant measure outside the cell, from somewhere on or inside the cell.

The term “transfection” means the introduction of a foreign nucleic acid into a cell. The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. The introduced gene or sequence may also be called a “cloned” or “foreign” gene or sequence, may include regulatory or control sequences, such as start, stop, promoter, signal, secretion, or other sequences used by a cell's genetic machinery. The gene or sequence may include nonfunctional sequences or sequences with no known function. A host cell that receives and expresses introduced DNA or RNA has been “transformed” and is a “transformant” or a “clone.” The DNA or RNA introduced to a host cell can come from any source, including cells of the same genus or species as the host cell, or cells of a different genus or species.

The term “host cell” means any cell of any organism that is selected, modified, transformed, grown, used or manipulated in any way, for the production of a substance by the cell, for example, the expression by the cell of a gene, a DNA or RNA sequence, a protein or an enzyme. Host cells can further be used for screening or other assays, as described infra.

The term “expression system” means a host cell and compatible vector under suitable conditions, e.g. for the expression of a protein coded for by foreign DNA carried by the vector and introduced to the host cell. Common expression systems include E. coli host cells and plasmid vectors, insect host cells and Baculovirus vectors, and mammalian host cells and vectors. In a specific embodiment, the protein of interest is expressed in COS-1 or C2C12 cells. Other suitable cells include CHO cells, HeLa cells, 293T (human kidney cells), mouse primary myoblasts, and NIH 3T3 cells.

### PAMPs

As noted above, PAMPs for use in the present invention activate TLR2 or TLR5. In a specific embodiment, exemplified below, a PAMP molecule that activates TLR5 elicited protective immunity against Listeria infection. Accordingly, the invention particularly relates to a PAMP specific for TLR5.

Bacterial lipoprotein (BLP) and bacterial outer membrane protein A (OmpA) represent two representative TLR2 specific PAMP molecules. For example, E. coli BLP (bacterial lipoprotein; Genbank Accession # X68953) can provide starting material for a vaccine construct of the invention. US published applications 2002/0131983 and 2003/0017162 also describe bacterial lipoproteins, in the context of traditional adjuvant molecules, which can be used in vaccine constructs of this invention; see also Published PCT application WO 96/32963. Other lipoproteins include, but are not limited to, the Oprl protein of P. aeruginosa; the fimbrial subunit protein of Porphyromonas gingivalis; the macrophage-activating lipopeptide 2 (MALP-2) of Mycoplasma fermentans; and the 19 kDa protein p19 from Mycobacterium tuberculosis. It is to be understood, however, that the scope of the present invention is not to be limited to any particular lipoprotein or lipoproteins. Outer membrane proteins include OmpA, E. coli OmpA with GenBank Accession numbers AAC74043.1 and AE000198.1.

Flagellin is a TLR5 activating PAMP (see, e.g., US published application 2002/0061312; see also US published application 2003/044429, the teachings of which are hereby incorporated by reference in its entirety). Exemplary flagellins include those identified with Genbank Accession numbers for the amino acid and nucleotide sequences of E. coli FliC (flagellin; Genbank Accession # AB028476) and of S. typhimurium fljB (flagellin; Genbank Accession # AF045151) for PAMP:antigen constructs. In addition to flagellin from S. typhimurium, flagellin for use in the invention also includes polypeptides from other bacterial species, such as H. pylori, V cholera, S. marcesens, S. flexneri, T. pallidum, L. pneumophilia; B burgdorferei; C. difficile, R. meliloti, A. tumefaciens; R. lupini; B. clarridgeiae, P. mirabilis, B. subtilus, L. monocytogenes, P. aeruginosa and E. coli (see, e.g., US published application 2003/044429, the teachings of which are hereby incorporated by reference in its entirety). A flagellin for use in the invention also includes flagellin polypeptides from other bacterial species.

### Polypeptide Antigens of L. monocytogenes

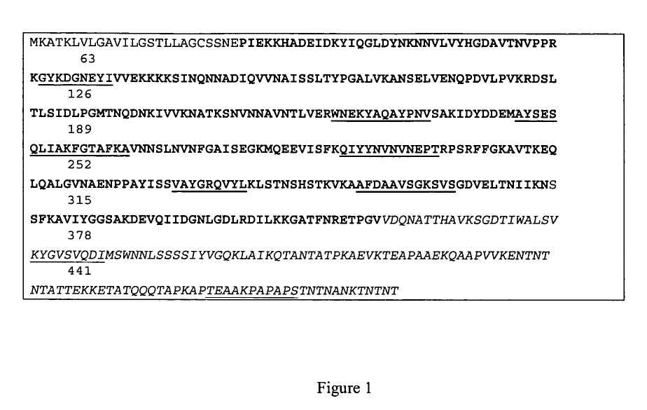

L. monocytogenes antigens p60 and LLO both have been shown to be protective in the mouse model of listeriosis. Accordingly, in a specific example, a combined fusion of p60 and LLO is used as the antigenic polypeptide in the vaccine construct (FIG. 1). In another embodiment, one or more of the individual T cell epitopes from either or both of the Listeria proteins that provide the antigenic polypeptides, e.g., as illustrated in Table 1 below, are provided in a specific construct.

TABLE 1

| | | | | MHC | SEQ |
|---|---|---|---|---|---|
| Mouse | | | | Restric- | ID |
| Strain | Epitope | Sequence | Type | tion | NO.: |
| BALB/c | LLO₉₁₋₉₉ | GYKDGNEYI | CD8 | K^{d} | 31 |
| | LLO₁₈₉₋₂₀₀ | WNEKYAQAYPNV | CD4 | A^{d} | 32 |
| | LLO₂₁₆₋₂₂₇ | QLIAKFGTAFKA | CD4 | A^{d} | 33 |
| | LLO₂₁₁₋₂₂₂ | AYSESQLIAKFG | CD4 | A^{d} | 34 |
| | p60₂₁₇₋₂₂₅ | KYGVSVQDI | CD8 | K^{d} | 35 |
| | p60₃₀₀₋₃₁₁ | TEAAKPAPAPST | CD4 | A^{d} | 36 |
| C57BL/6 | LLO₁₉₀₋₂₀₁ | NEKYAQAYPNVS | CD4 | A^{b} | 37 |
| | LLO₃₁₈₋₃₂₉ | AFDAAVSGKSVS | CD4 | A_{b} | 38 |
| | LLO₂₉₆₋₃₀₄ | VAYGRQVYL | CD8 | K^{b} | 39 |
| | LLO₂₉₇₋₃₀₄ | AYGRQVYL | CD8 | K^{b} | 40 |
| | LLO₂₅₃₋₂₆₄ | QIYYNVNVNEPT | CD4 | A^{b} | 41 |

Table 1: T-cell epitopes of LLO and p60 contained in the P2.LIST and STF2.LIST constructs, and their MHC restriction elements in BALB/c and C57BL/6 mice. LLO₉₁₋₉₉ (Pamer et al, op cit.); p60₂₁₇₋₂₂₅ (Pamer et al, op cit.); p60₃₀₀₋₃₁₁ (Geginat et al, 1998); all others (Geginat et al, 2001).

### Immunogenicity Assays

The vaccine constructs of the invention are potent immunogens, able to elicit protective immunity from Listeria infection. Various in vitro and in vivo (animal model) assays demonstrate the immunogenic potential of the vaccine constructs. For example, as fusion proteins are purified, their biological activity can be characterized in an NF-κB activation assay. Since TLRs signal through NF-κB, cells stably transfected with an NF-κB luciferase reporter construct are used to confirm biological activity of PAMP:antigen fusion proteins. Cell lines used in this assay may constitutively express the appropriate TLR, or may be engineered to overexpress the TLR of choice. Cells seeded in a 96-well microplate (could be 384 or 1536-well) are exposed to test compounds for four to five hours at 37° C. NF-κB-dependent luciferase activity may be measured using readily available systems, such as the Steady-Glo Luciferase Assay System by Promega (E2510), following the manufacturer's instructions. Luminescence can be measured on a microplate luminometer, e.g., FARCyte (Amersham). Specific activity of test compound can be expressed as the EC₅₀, i.e., the concentration which yields a response that is 50% of the maximal response obtained with the appropriate control reagent, such as peptidoglycan (TLR2 ligand).

In addition, dendritic cell maturation assays demonstrate activity. For example, DCs can be generated in vitro as previously described (Lutz et al., J. Immol. Meth. 1998, 223:77-92). In brief, bone marrow cells from 6-8 week old C57BL/6 mice are isolated and cultured for 6 days in medium containing 10% FCS and 100 U/ml GMCSF, replenishing half the medium every two days. On day 6, nonadherant cells are harvested and resuspended in medium without GMSCF. Test compounds are added and the cultures are incubated for 16 hours. Supernatants are harvested, and cytokine concentrations are determined by sandwich enzyme-linked immunosorbent assay (ELISA) using matched antibody pairs from BD Pharmingen or R&D Systems, following the manufacturer's instructions. Cells are harvested, and expression of MHC Class II and costimulatory molecules (e.g., B7-2) can be determined by flow cytometry using antibodies from BD Pharmingen or Southern Biotechnology Associates and following the manufacturer's instructions. DCs can also be resuspended in staining buffer and blocked with anti-FcR mab (2.4G2), followed by staining with fluorescently-labeled mAbs to CD11c (HL-3), MHC Class II (2G9), CD80 (16-10A1), and CD86 (GL1) (all from BD). Cell surface expression of determinants is assessed using a flow cytometer and appropriate data capture and analysis software, e.g., Becton Dickinson FACscan and Cellquest software.

In addition, one can perform a cytokine analysis and chemokine analysis, including secretion of IFN-gamma, TNF-alpha, IL-12 p70, IL-10 and IL-6 following stimulation of DCs with fusion protein. Cytokine content of stimulated DC culture supernatant is determined by ELISA using matched antibody pairs (BD Pharmingen) and following the manufacturer's instructions. Intracellular cytokine expression is determined by flow cytometry on fixed cells as previously described (Serbina et al., Immunity 2003 19:59-70). Analysis can be done on a flow cytometer and appropriate data capture and analysis software, e.g., BD FACscan running Cellquest software. Expression of chemokine genes can be monitored by RNAse protection assay using the Riboquant Assay system from BD according to the manufacturer's instructions. The mCK-c6, mCK-3b and mCK-5b templates allow analysis of expression of Rantes, MIP-1, MIP-2, MCP-1, CCR1, CCR2, CCR3, CCR5, and CCR7.

Ex vivo studies include testing protective immunity to L. monocytogenes. Such tests can involve the induction of antigen specific cellular and humoral responses. The nature and magnitude of these responses can be measured following vaccination with a vaccine construct. At specific time points following vaccination (i.e., day 7, 14, 30, 120), animals will be examined for antigen-specific humoral and cellular responses, including serum antibody titers, cytokine expression, CTL frequency and cytotoxicity activity, and antigen-specific proliferative responses.

For example, one can measure induction of antigen-specific T cell responses. CD8 T cell responses are monitored by quantitating the number of antigen-specific gamma-interferon secreting cells using ELISPOT (R&D Systems). At varying time point post-vaccination, T cells are isolated from the draining lymph nodes and spleens of immunized animals and cultured in microtiter plates coated with capture antibody specific for the cytokine of interest. Synthetic peptides corresponding to the H-2K^{d}-restricted epitopes, p60₂₁₇₋₂₂₅ and LLO₉₁₋₉₉ are added to cultures for 16 hours. Plates are washed and incubated with anti-IFN-gamma detecting antibodies as directed by the manufacturer. Similarly, CD4 responses can be quantified by IL4 or IL-5 ELISPOT following stimulation with the I-A^{d} restricted CD4 epitopes LLO₁₈₉₋₂₀₀, LLO₂₁₆₋₂₂₇, and p60₃₀₀₋₃₁₁. Antigen specific responses can be quantified using a dissection microscope or a microplate spectrophotometer equipped to identify and enumerate discrete spots, e.g., Immunospot 3A from Cellular Technologies Ltd. For quantitation of CD8 responses, it is also possible to utilize flow cytometric analysis of T cell populations following staining with recombinant MHC Class I tetramer loaded with the H-2^{d} restricted epitopes noted above.

Induction of antigen-specific CTL activity can be measured following in vitro restimulation of lymphoid cells from immune and control animals, using a modification of the protocol described by Bouwer and Hinrichs (Bouwer et al., Infect. Immun. 1996, 64:2515-2522). Briefly, erythrocyte-depleted spleen cells are cultured with Concanavalin A or peptide-pulsed, mitomycin C-treated syngeneic stimulator cells for 72 hours. Effector lymphoblasts are harvested and adjusted to an appropriate concentration for the effector assay. Effector cells are dispensed into round bottom black microtiter plates. Target cells expressing the appropriate antigen (e.g., cells infected with live L. monocytogenes or pulsed with p60 or LLO epitope peptides) are added to the effector cells to yield a final effector:target ratio of at least 40:1. After a four hour incubation, target cell lysis is determined by measuring the release of LDH using the CytoTox ONE fluorescent kit from Promega, following the manufacturer's instructions.

Antigen-specific T-cell proliferation and cytokine secretion can be measured following in vitro restimulation of lymphoid cells from immune and control animals following standard protocols. Briefly, erythrocyte-depleted draining lymph node and spleen cells are cultured in replicate wells of round-bottom plates with peptide-pulsed, mitomycin C-treated syngeneic stimulator cells for three to four days at 37° C. Supernatants are harvested and frozen for later analysis of cytokine concentrations. Proliferation in the cultures is determined using the CellTiter 96 Aqueous One Solution kit from Promega, following the manufacturer's instructions, or incorporation of ³H-thymidine. Cytokine concentrations in the supernatants are determined by sandwich ELISA according to manufacturer's directions.

Antigen-specific antibody titers can be measured by ELISA according to standard protocols (Cote-Sierra et al), with modifications. Alternatively, antigen-specific antibodies of different isotypes can be detected by Western blot analysis of sera against lysates of whole L. monocytogenes, using isotype-specific secondary reagents.

### Protection Against Listeriosis

The challenge models described above provide a basis for determining the effects of variations in dose and regimen, and, using routine dose-response studies, to identify the minimal dose required to protect 100% of the test animals, the regimen (minimal number of inoculations) required to protect 100% of the test animals, and the longevity of the memory response induced by the minimal effective dose and regimen. For example, mice can be immunized with a single inoculation of 1 μg, 5 μg, 10 μg, or 20 μg per mouse prior to challenge to determine the minimal effective dose. Immunizations at each dose level are be done in single inoculations and repeat inoculations in different groups of mice to determine whether repeat inoculation confers a higher incidence of protection, or protection against a more robust challenge dose. To examine the longevity of the protective response, mice can be challenged with live L. monocytogenes at various time points (e.g., 7, 28, 60, and 90 days) after immunization and evaluated for splenic and liver bacterial burdens 3 days post-infection.

### Prevention of L. monocytogenes Infection

In yet another aspect of the present invention, pharmaceutical compositions of the above vaccine construct compounds are provided for immunoprotection against Listeriosis. Delivery can be of the vaccine construct as a protein. If efficacious, delivery can also be of the DNA form of the vaccine construct. Such pharmaceutical compositions may be for administration by oral, injectable (intramuscular, intraperitoneal, intravenous (IV) or subcutaneous injection), transdermal (either passively or using iontophoresis or electroporation), transmucosal (nasal, vaginal, rectal, or sublingual) routes of administration or using bioerodible inserts and can be formulated in dosage forms appropriate for each route of administration. The dosage of administration for such a vaccine will vary depending upon the composition, method of delivery, and animal administered, and will be experimentally derived. In general, comprehended by the invention are pharmaceutical compositions comprising effective amounts of an vaccine construct, or derivative products, of the invention together with pharmaceutically acceptable diluents, preservatives, solubilizers, emulsifiers, adjuvants and/or carriers. Such compositions include diluents of various buffer content (e.g., Tris-HCl, acetate, phosphate), pH and ionic strength; additives such as detergents and solubilizing agents (e.g., Tween 20, Tween 80, Polysorbate 80), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc. or into liposomes. Hylauronic acid may also be used. Such compositions may influence the physical state, stability, rate of in vivo release, and rate of in vivo clearance of the present proteins and derivatives. See, e.g., Remington's Pharmaceutical Sciences, 18th Ed. (1990, Mack Publishing Co., Easton, Pa. 18042) pages 1435 1712 which are herein incorporated by reference. The compositions may be prepared in liquid form, or may be in dried powder (e.g., lyophilized) form.

Injectable delivery: Preparations according to this invention for injectable administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized by, for example, filtration through a bacteria retaining filter, by incorporating sterilizing agents into the compositions, by irradiating the compositions, or by heating the compositions. They can also be manufactured using sterile water, or some other sterile injectable medium, immediately before use.

The present invention is further illustrated by the following examples, which are not intended to be limiting in any way.

### EXEMPLIFICATION

### Example 1

### PAMP.LIST Constructs

### Materials and Methods

Sequences encoding the signal sequence and lipidation motif of E. coli lipoprotein (SEQ ID NO: 2; amino acid 1-24; designated P2), Salmonella typhimurium flagellin fljB (SEQ ID NO: 6; designated STF2), E. coli outer membrane protein A (SEQ ID NO: 4) and L. monocytogenes LLO₄₈₋₃₇₉ (SEQ ID NO: 10) and p60₁₉₃₋₃₁₉ (SEQ ID NO: 9) were cloned by employing sets of primers derived from the published sequences of the various using genomic bacterial DNA as the template in a PCR reaction. The flagellin sequences of this construct were isolated from S. typhimurium genomic DNA by PCR using the following primers: forward (5′ ATG GCA CAA GTA ATC AAC ACT AAC 3′ (SEQ ID NO.: 22) and reverse (5′ CTC GAG ACG TAA CAG AGA CAG CAC GTT CTG 3′ (SEQ ID NO.: 23). Primers used for the amplification of OmpA are as follows: ECOMPA-F: 5′ AATGAAAAAGACAGCTATCGCGATTGCAGTGGCACTG-3′ (SEQ ID NO.: 24) (forward) and OMBHD-R2: 5′ AAGCTTCGAATTGCCCTTAGCCTGCGGCTGAGTTACAACG-3′ (SEQ ID NO.: 25) (reverse).

For L. monocytogenes, first primers LLOF7 forward (LLOF7: 5′-CTTAAAGAATTCCCAATCGAAAAGAAACACGCGGATG-3′ (SEQ ID NO.: 26) and LLOR3 reverse (LLOR3: 5′-TTCTACTAATTCCGAGTTCGCTTTTACGAG-3′ (SEQ ID NO.: 27) were used to amplify a 5′ portion of the LLO sequences. Next primers LLOF6 forward (LLOF6: 5′-CTCGTAAAAGCGAACTCGGAATTAGTAGAA-3′ (SEQ ID NO.: 28) and P60R7 reverse (P60R7: 5′ AGAGGTCTCGAGTGTATTTGTTTTATTAGCATTTGTG-3′ (SEQ ID NO.: 29) were used to amplify the remaining fused 3′ portion LLO sequences and the P60 sequences. These two PCR fragments were then joined by a third PCR using the primers LLOF7 and P60R7. This PCR served to mutate the LLO sequence spanned by LLOR3 and LLOF6 so as to remove the EcoRI site. This product was then ligated into the vector P2/pCRT7CT, to yield P2LIST in which the 1.3 kb Listeria sequences insert is flanked at the 5′ end with the signal sequence and lipidation motif from bacterial lipoprotein and at the 3′ end has a 6× histidine epitope tag. The PCR products were ligated into pCR2.1-TOPO cloning vector (Invitrogen). In this vector, the chimeric DNA insert is driven by the strong T7 promoter, and the insert is fused in frame to the V5 epitope (GKPIPNPLLGLDST) (SEQ ID NO.: 30) and polyhistidine (6×His) is located at the 3′ end of the gene. The identity and size of the inserts were determined by restriction enzyme mapping and verified by DNA sequencing.

A P1-cassette was made in pET23a(+) by cloning a PCR product derived from full-length BLP using the primers: BLPF8 5′-TTAGTCCATATGAAAGCTACTAAACTGGTACTG-3′ (SEQ ID NO: 43)

BLPR9: 5′-AAGATTGAATTCGCGGTATTTAGTAGCCATGTTG-3′ (SEQ ID NO: 44) and subcloning the fragment into NdeI and EcoRI sites of the vector.

To make the P2 cassette for cloning of LLO-p60 (LIST) sequences, a portion of BLP was derived from the full-length cassette, P1 (see below) using the primers: P23F1: 5′-ACCGTCATCACCGAAACG-3′ (SEQ ID NO: 45)

BLPR10 5′-AACTAAGAATTCGTTGCTGGAGCAACCTGCCAG-3′ (SEQ ID NO: 46). P23F1 is upstream of a PvuII site and BLPR10 has an EcoRI site. The PCR product was cloned this into those sites of PET23a(+). The P2 cassette was inserted into the cloning sites of EcoRI and XhoI for insertion of LIST fragment derived from PCR. Before cloning in LIST, a silent mutation was made in a 5′ EcoRI site of the LIST epitope. P1 cassette was made in pET23a(+), by cloning PCR product comprising the full-length BLP using the following primers: BLPF8: 5′-TTAGTCCATATGAAAGCTACTAAACTGGTACTG-3′ (SEQ ID NO: 43)

BLPR9: 5′-AAGATTGAATTCGCGGTATTTAGTAGCCATGTTG-3′ (SEQ ID NO: 44) using NdeI and EcoRI sites in the vector.

In order to construct STF2.LIST, a forward primer (5′-GTCTCGAGGAATTCCCAATCGAAAAGAAACACGCG-3′ (SEQ ID NO: 47)) and a reverse primer (5′-ACGGCACTGGTCAACTTGGCCATGGTG-3′ (SEQ ID NO: 48)) were used to in a PCR amplification on P2.LIST DNA as template. The 1.4 kb PCR product was ligated by cohesive ends to STF2.TRP2 vector that was prepared by deleting the TRP2 gene. Positive colonies were identified by PCR screening using vector specific primers and by restriction mapping. The resulting constructs were confirmed by DNA sequencing. The STF2.LIST harbors both V5 and polyhistidine tags at the C-terminus of the fusion

### Results and Discussion

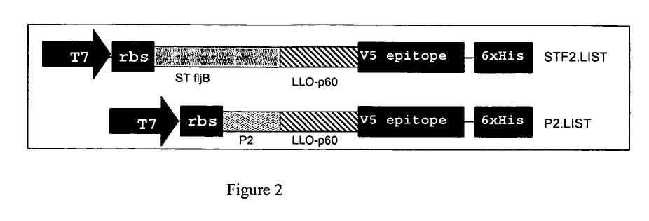

The constructs described in Table 2 and FIG. 2 were derived by standard molecular biological techniques. The predicted molecular weights for P2.LIST (SEQ ID NOS.: 11 and 12) and STF2.LIST (SEQ ID NOS.: 13 and 14) are about 55 Kd and about 107 K_{d}, respectively

TABLE 2
PAMP: Listeria antigen fusion constructs

| Construct | PAMP | Antigen |
|---|---|---|
| LIST | None | Listeria LLO and p60 |
| STF2 | Salmonella fljB | None |
| STF2.LIST | Salmonella fljB | Listeria LLO and p60 |
| P2.LIST | E. coli BLP | Listeria LLO and p60 |

### Example 2

### Protein Expression, Purification and Confirmation

### Materials and Methods

Protein expression and immunoblot assay: E. coli strain BL21 (DE3) pLysS (Invitrogen) was transformed with DNA purified from P2.LIST and STF2.LIST using a commercially available kit (Qiagen). A colony was inoculated into 2-ml LB containing 100 μg/ml carbenicillin, 34 μg/ml chloramphenicol supplemented with 0.5% glucose and grown overnight at 37° C. with shaking. A fresh 2-ml culture was inoculated with a 1:20 dilution of the overnight culture and grown at 37° C. for several hours until OD₆₀₀=0.5-0.8. Protein expression was induced by the addition of IPTG to 1 mM for 3 hours. The bacteria were harvested by centrifugation and the pellet was re-suspended in 100 μl of 1×SDS-PAGE sample buffer in the presence of β-mercaptoethanol. The samples were boiled for 5 minutes and 1/10 volume of each sample was loaded onto 10% SDS-PAGE gel and electrophoresed. The samples were transferred to PVDF membrane and probed with antibodies specific for polyhistidine (Tetra His, Qiagen) at 1:1000 dilution and rabbit anti-mouse IgG/AP conjugate (Pierce) at 1:25,000 as secondary antibody. The immunoblot was developed using BCIP/NBT colometric assay kit (Promega).

Large-scale protein purification: E. coli cells transformed with the construct of interest were cultured and induced as described above. Cells were harvested by centrifugation at 7,000 rpm for 7 minutes at 4° C. in a Sorvall RC5C centrifuge. The cell pellet was resuspended in Buffer A (6 M guanidine HCl, 100 mM NaH₂PO₄, 10 mM Tris-HCl, pH 8.0). The suspension can be frozen at −80° C. if necessary. Cells were disrupted by passing through a microfluidizer at 16,000 psi. The lysate was centrifuged at 30,000 rpm in a Beckman Coulter Optima LE-80K Ultracentrifuge for 1 hour. The supernatant was decanted and applied to Nickel-NTA resin at a ratio of 1 ml resin/1 L cell culture. The clear supernatant was incubated with equilibrated resin for 2-4 hours by rotating. The resin was washed with 200 volumes of Buffer A, followed by a subsequent wash with 200 volumes of Buffer B (8 M urea, 100 mM NaH₂PO₄, 10 mM Tris-HCl, pH 6.3) to remove non-specifically bound proteins. An additional 200 volume wash with buffer C (10 mM Tris-HCl, pH 8.0, 60% isopropanol) reduced endotoxin to acceptable level (less than 0.1 EU/μg). Protein was eluted with Buffer D (8 M Urea, 100 mM NaH₂PO₄, 10 mM Tris-HCl, pH 4.5). Protein elution was monitored by SDS-PAGE, Western blot (anti-His, anti-LLO and anti-p60), and endotoxin level was determined by LAL assay (described below). If endotoxin level remained unacceptably high, the protein was chromatographed through Superdex 200 gel filtration in the presence of 1% deoxycholate to separate protein and endotoxin. A second round of Superdex 200 gel filtration in the absence of deoxycholate removed the detergent from the protein sample. Purified protein was concentrated and dialyzed against 1×PBS, 1% glycerol. The protein was aliquoted and stored at −80° C.

### Results and Discussion

Fusion proteins were expressed with a 6× Histidine tag to facilitate purification. The general large-scale purification protocol is outlined as follows: A 12 L culture is grown, cells are harvested and lysed in buffer A, the cleared lysate purified by Ni-NTA chromatography which binds the 6× Histidine tag, washed with buffers A, B, and C, and eluted with buffer D. By this method, expression and immunoblot detection of the 55 Kd recombinant P2.LIST and 107 Kd STF2.LIST fusion protein in E. coli at greater than 95% purity was achieved. Table 3 describes typical batch characteristics of P2.LIST and STF2.LIST (also referred to herein as “STF2 and LLO-p60”) purified as described. Purity was determined by SDS-PAGE. Endotoxin level was determined by the LAL assay. NF-κB and DC assays are described in in vitro assays for PAMP activity, below. This protocol or modifications thereof are used for selection and expression of additional constructs as needed.

TABLE 3
Summary of Final Products

| | Protein | | Endotoxin | Functional Assay | |
|---|---|---|---|---|---|
| | Concentration | | Level | NF-κB | DC |
| Protein | (μg/ml) | Purity | (EU/μg) | Assay | Assay |
| P2.LIST | 200 | 99% | 0.08 | + | + |
| STF2.LIST | 250 | 99% | 0.06 | + | + |

### Example 3

### Analysis of Proteins & Confirmation of Activity

### Materials and Methods

Cell lines expressing TLR and luciferase reporter: 293, 3T3, and RAW cells were assayed by RT-PCR to determine endogenous expression of Toll-like receptors 2, 4, and 5. For each cell line, an NF-κB reporter vector containing tandem copies of the NF-κB consensus sequence upstream of a minimal promoter fused to the firefly luciferase gene was co-transfected with a vector containing an antibiotic resistance gene for selecting stable clones. 293luc (fibroblast, TLR expression 2⁻4⁻5⁺), 3T3κB (fibroblast, TLR expression 2⁺4⁺5⁻) and RAWκB (macrophage, TLR expression 2⁺4⁺5⁻) cell lines are utilized to measure NF-κB dependent luciferase activity triggered by the activation of specific Toll-like receptors 5, 4, and 2, respectively. When NF-κB transcription factors produced by the activation of Toll-like receptors bind to the cis-acting enhancer element in the reporter vector, transcription is induced and luciferase is made.

NF-κB activation assay: Cells (100 μl volume) seeded in a 96-well microplate were exposed to 10 μl of large-scale preparations of purified P2.LIST or STF2.LIST (final concentration about 0.001 to 10 μl/ml) for four to five hours at 37° C. NF-κB-dependent luciferase activity was measured using the Steady-Glo Luciferase Assay System by Promega (E2510), following the manufacturer's instructions. Luminescence was measured on a microplate luminometer (FARCyte, Amersham). Specific activity of test compound was expressed as the EC₅₀, i.e., the concentration which yields a response that is 50% of the maximal response obtained with the appropriate control reagent, such as peptidoglycan (TLR2 ligand) or STF2.OVA (TLR5 ligand).

Dendritic cell maturation: DCs were generated in vitro as previously described (Lutz et al, 1998). In brief, bone marrow cells from 6-8 week old C57BL/6 mice were isolated and cultured for 6 days in medium containing 10% FCS and 100 U/ml GMCSF, replenishing half the medium every two days. On day 6, nonadherant cells were harvested and resuspended in medium without GMSCF. Cells (100 μl volume) seeded in a 96-well microplate were exposed to 10 μl of largescale purified preparations of purified P2.LIST or STF2.LIST (final concentration about 0.001 to 10 μl/ml) were added and the cultures were incubated for 16 hours. Cells were harvested, and expression of MHC Class II or B7-2 was determined by flow cytometry using antibodies from BD Pharmingen following the manufacturer's instructions.

Endotoxin assay: Endotoxin levels in recombinant fusion proteins were measured using the QCL-1000 Quantitative Chromogenic LAL test kit (BioWhittaker #50-648U), following the manufacturer's instructions for the microplate method.

### Results and Discussion

In vitro assays were performed to determine the PAMP activity of purified proteins. Since TLRs signal through NF-κB, cells stably transfected with an NF-κB luciferase reporter construct were used to confirm biological activity of PAMP:antigen fusion proteins. Using reporter cell lines that can respond specifically to signaling via TLR2 and 5, the TLR specificity of specific recombinant fusion proteins can be determined. As demonstrated in Table 3, supra, typical batches of P2.LIST and STF2.LIST purified as described display low levels of endotoxin. Furthermore, P2.LIST and STF2.LIST activated luciferase activity in RAWκB (TLR2+) cell and 293luc (TLR5+) cell, respectively, and MHC Class II expression was confirmed in the dendritic cell maturation assays, demonstrating the specific TLR signaling activity of the proteins.

### Example 4

### Comparison of Efficacy of Recombinant Proteins in Murine Models of Listeriosis

### Materials and Methods

Immunization: Recombinant fusion protein was suspended in phosphate-buffered saline (PBS), without exogenous adjuvant. BALB/c (n=10-20 per group) mice were immunized intraperitoneally with PBS, STF2.OVA (30 μg) or STF2.LIST (30 μg). Positive control animals were immunized with 10³ CFU of live L. monocytogenes, while negative control animals received mock-immunization with PBS alone.

Sublethal L. monocytogenes challenge: Seven days after immunization, BALB/c mice were infected by intravenous injection of 5×10³ CFU of live L. monocytogenes (strain 10403s) in 0.1 ml of PBS. Three days following challenge, bacterial burden in the spleens was determined. Spleens and livers were removed 72 hours after infection and homogenized in 5 ml of sterile PBS+0.05% NP40. Serial dilutions of the homogenates were plated on BHI agar. Colonies were counted after 48 hours of incubation. These experiments were performed a minimum of 3 times utilizing 10-20 animals per group. Mean bacterial burden per spleen or liver was compared between treatment groups by Student's t-test.

### Results and Discussion

The mouse model of listeriosis (Geginat et al, op cit.) has provided insights into the mechanisms of disease and the immunological response to infection with L. monocytogenes. This model permits the study of both short-term and memory responses, and cellular and humoral compartments responsible for protection. This model was employed to examine the efficacy and mechanism of action of the novel PAMP:antigen fusion constructs of the present invention.

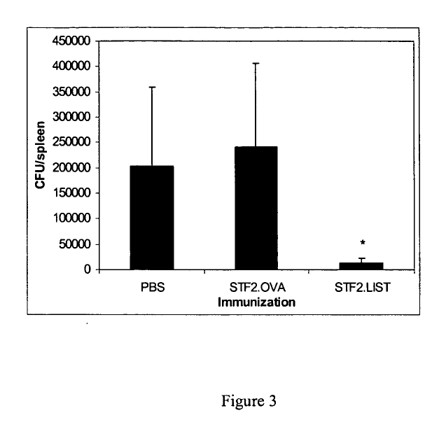

The data shown in FIG. 3 demonstrate a potent and significant protective effect of immunization with STF2.LIST against Listera monocytogenes. Spleens were harvested and bacterial burden was determined by quantitating the colony-forming units in each spleen. Data represent the mean±SD of five animals per group. *94% reduction, P=0.05 vs PBS; 95% reduction, P=0.04 vs STF.OVA. It is important to note that immunization with STF2.OVA did not protect from challenge with wild-type L. monocytogenes, as this organism does not express OVA. Thus, the protective effect was antigen-specific, not PAMP-specific.

### Example 5

### STF2.OVA Triggers a Characteristic Antigen Specific Response

### Materials and Methods

Constructs: STF2 was cloned from Salmonella typhimurium flagellin fljB as described in Example 1, supra. It was fused in frame with the model antigen chick ovalbumin (OVA).

Protein expression was as described in Example 2, supra, analysis of proteins and confirmation of activity as described in Example 3, supra, and murine challenge models as described in Example 4, supra.

Specifically, animals were immunized with STF2.OVA, OVA emulsified in CFA (CFA/OVA) or PBS as controls. Seven days following immunization sera were harvested and examined for OVA-specific IgG1 and IgG2a responses.

### Results and Discussion

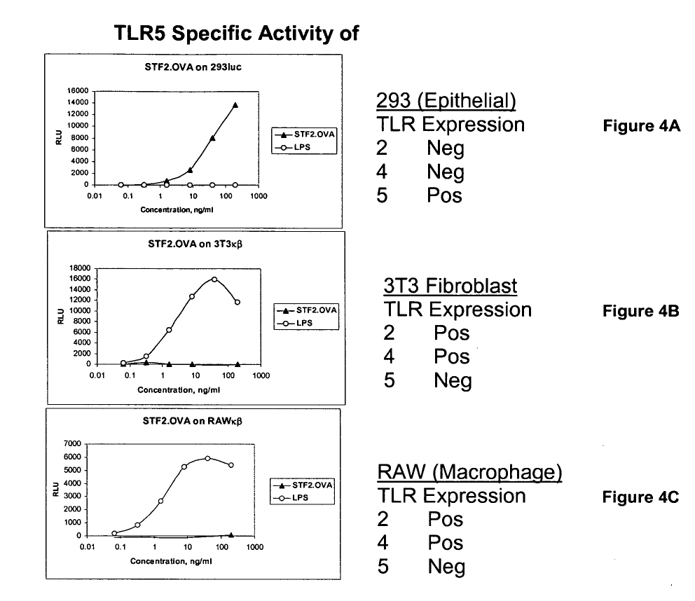

A recombinant protein consisting of full-length flagellin (S. typhimurium fljB, or STF2, a TLR5 ligand) fused to full-length ovalbumin (STF2.OVA) was produced and purified. The TLR5 specificity of STF2.OVA was examined in vitro by measuring the induction of NF-κB dependent luciferase activity in 293, 3T3 and RAW cells (FIG. 4). Results demonstrate the TLR5 specificity of STF2.OVA.

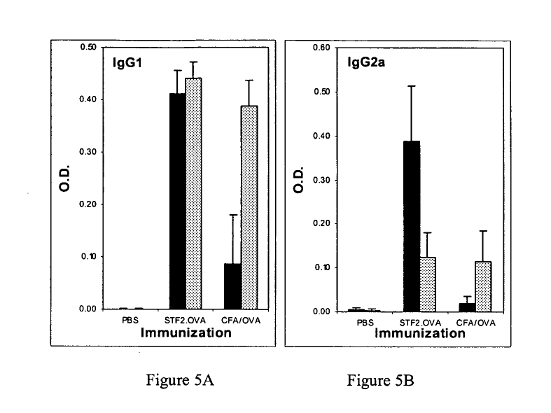

Animals immunized with STF2.OVA produced higher levels of OVA-specific IgG1 and IgG2a than animals immunized with CFA/OVA (FIG. 5 solid bars). A parallel group of animals was challenged by oral administration of LM.OVA, and antibody responses to OVA were examined five days post-challenge. Although no responses were detectable in animals immunized with PBS alone, STF2.OVA-immunized animals produced levels of OVA-specific IgG1 and IgG2a similar to those observed in animals receiving OVA emulsified in CFA (FIG. 5 stipple bars).

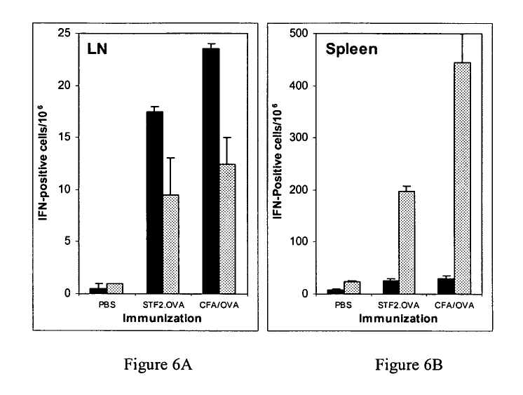

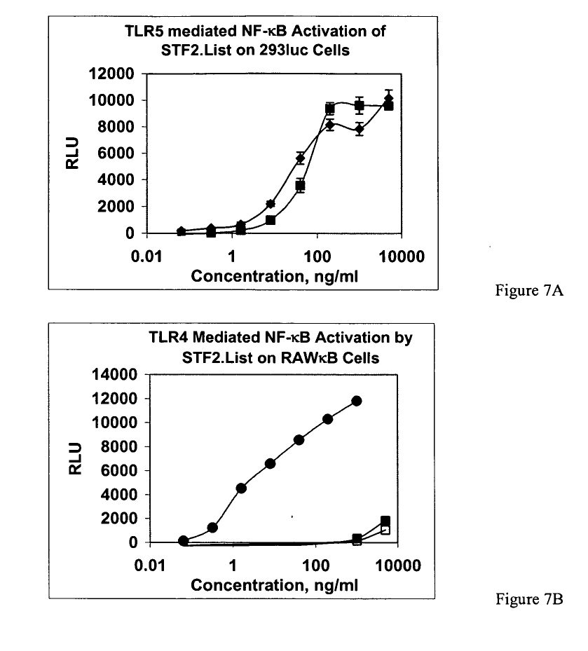

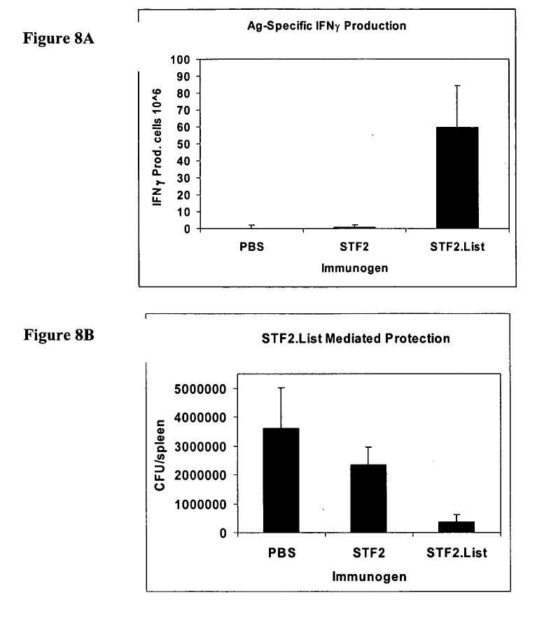

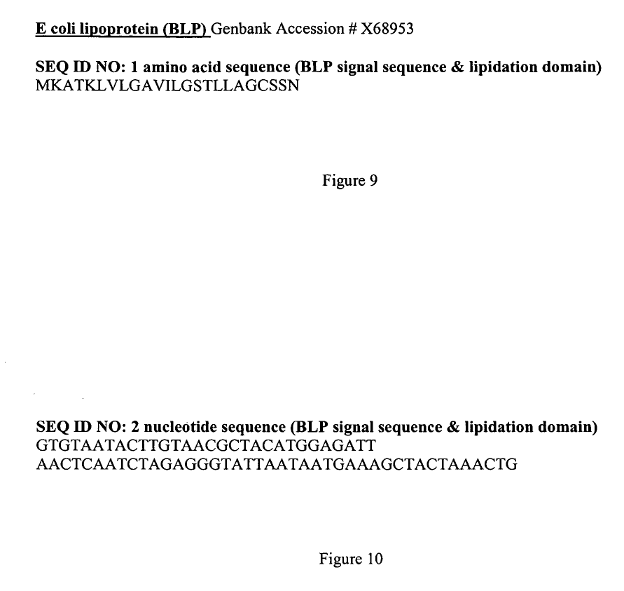

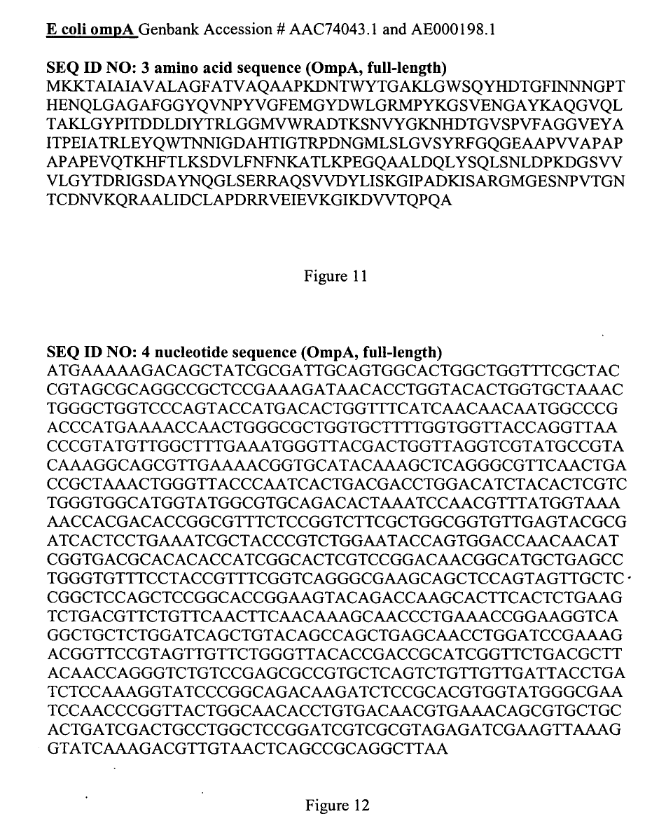

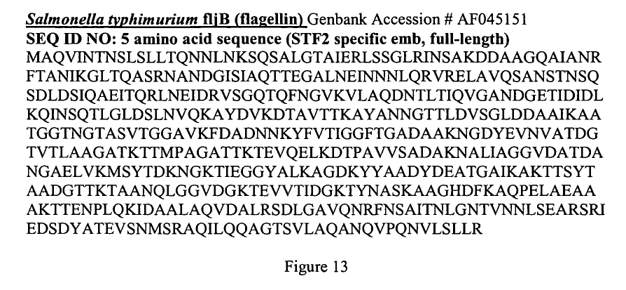

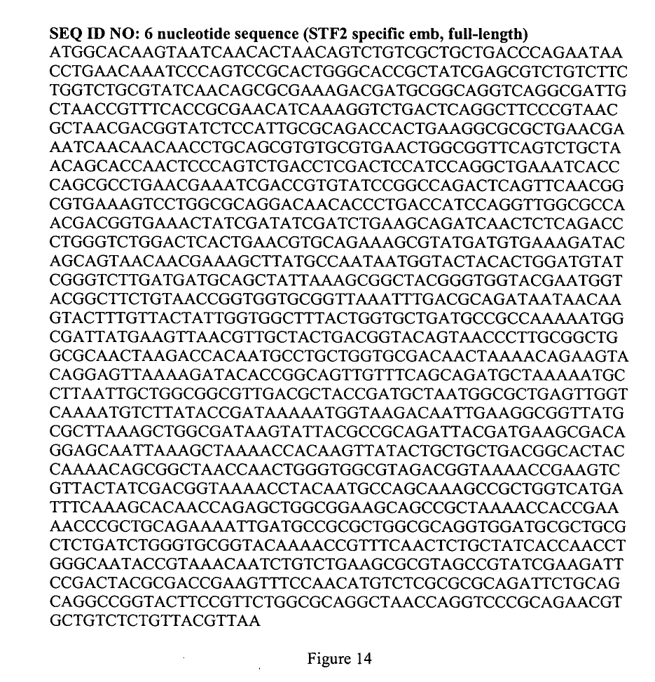

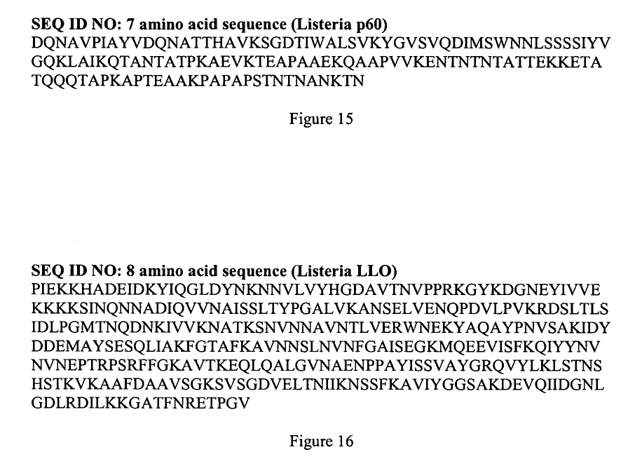

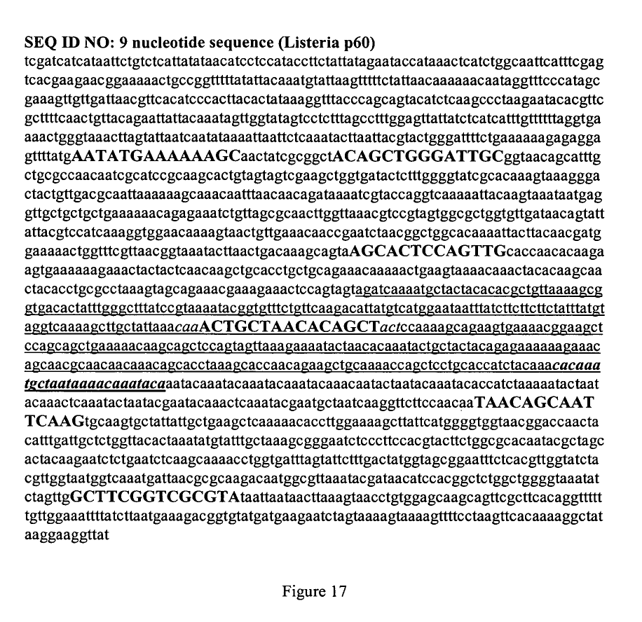

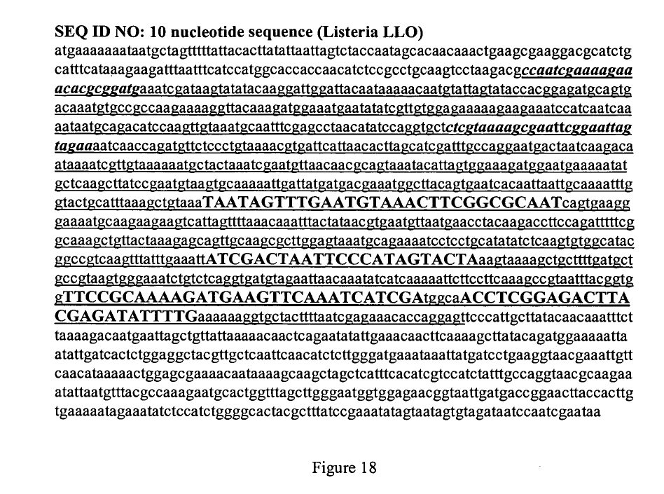

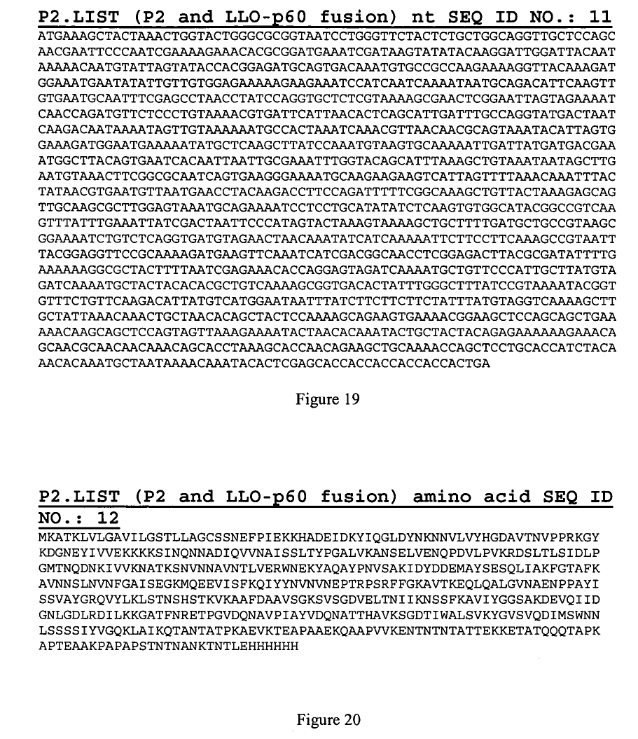

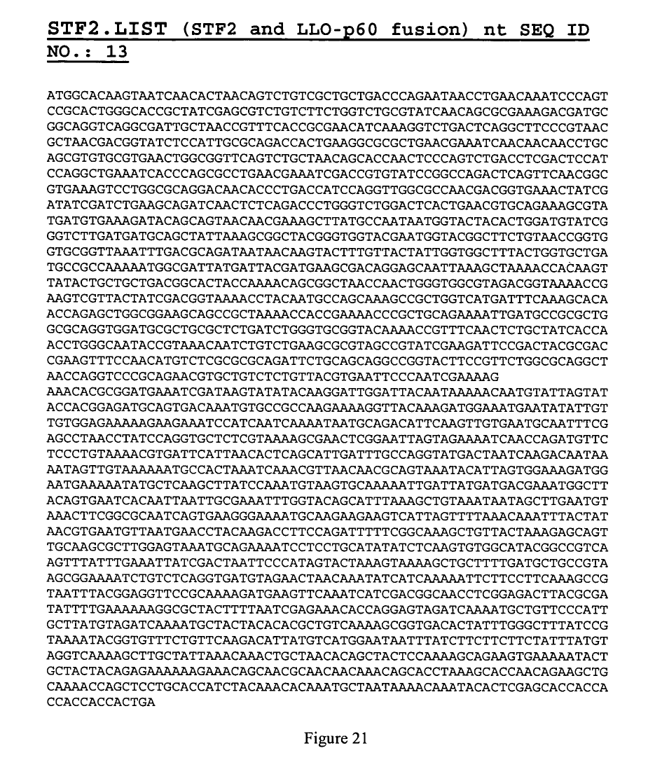

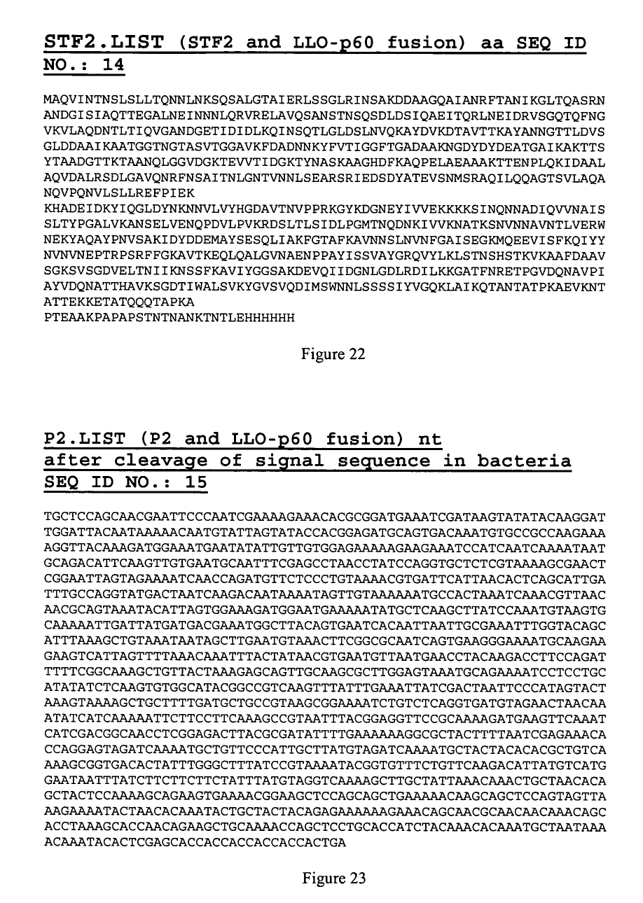

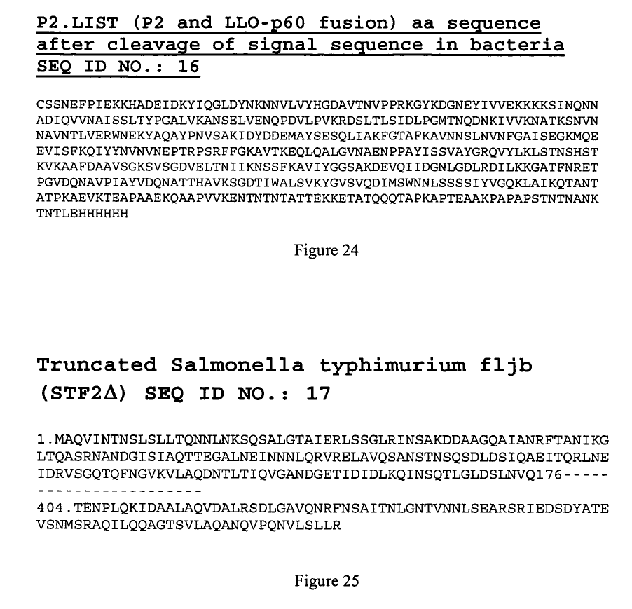

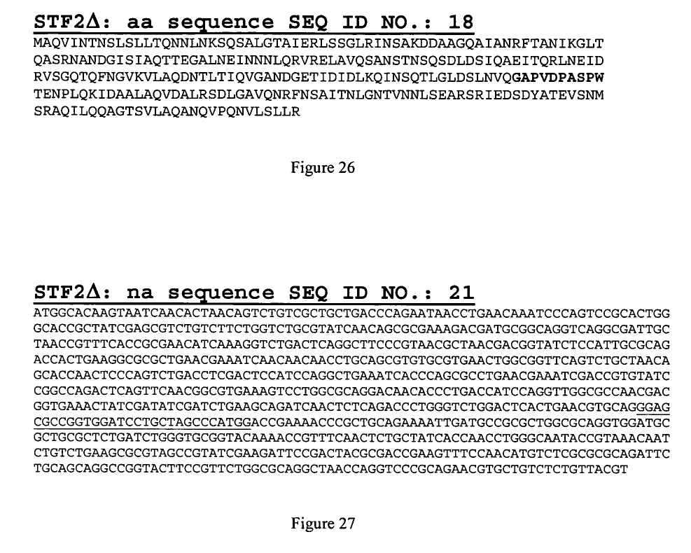

Draining lymph nodes cells of the mice depicted in FIG. 5 were restimulated in vitro with the H-2K^{d}-restricted OVA-derived peptide SIINFEKL, and the number of antigen-specific T cells was evaluated by gamma interferon ELISPOT. As shown in FIG. 6 (solid bars), the primary immune response to antigen induced by STF2.OVA was comparable to that induced by CFA/OVA. Interestingly, in both treatment groups the number of IFN-positive cells in the spleen increased significantly following challenge with LM.OVA (FIG. 6 Right). These data probably reflect homing of the antigen-specific T-cells to the spleen, which acts as a reservoir for L. monocytogenes infection.

Thus, STF2.OVA stimulated both T and B cell responses specific for the cognate antigen, OVA. This characteristic IgG specific and CD8 response demonstrates the ability of an antigen conjugated to Salmonella tyhpimurium flagellin fljB and purified by the methods utilized to induce a response characteristic of the TLR5 signaling pathway, capable of clearing a bacterial burden.

In comparing the immunogenicity of the STF2.OVA fusion protein versus mixture of STF2 plus OVA, sera from C57BL/6 mice receiving a single subcutaneous immunization with STF2.OVA showed significantly higher levels of IgG2a and IgG1 at seven days as compared to mice immunized with STF2, OVA, PBS, or STF2+OVA. These results demonstrate that a physical linkage of PAMP and antigen are required for optimal immunogenicity in vivo. STF2.OVA induced a dose-responsive antigen-specific cellular response in vivo as assayed by lymph node proliferative response. In addition, STF2.OVA provides an antigen-specific T cell response in vivo as assayed by IFNγ ELISPOT measurement.

### Example 6

### Construction of STF2Δ

To construct the STF2Δ, a portion of the (amino acids 176-404 of SEQ ID NO: 5 encoded by nucleic acids 526-1212 of SEQ ID NO: 6) the hypervariable region (amino acids 170-415, of SEQ ID NO: 5) of fljB (STF2) was deleted. The STF2Δ delection leaves the amino-terminus and carboxy-terminus domains of STF2 in tact. In the first PCR the following primers were combined with pCRT7/STF2 as DNA template in a PCR amplification: using the following forward and reverse primers:

STF28BGF-1:

(SEQ ID NO.: 31)

5′CTCGGGAGATCTGCACAAGTAATCAACACTAACAGTCT-3′
STF28MCR-1:

(SEQ ID NO.: 32)

5′CCATGGGCTAGCAGGATCCACCGGCGCTCCCTGCACGTTCAGTGAGTC
CAGACCCAGGG-3′.

In another set of PCR was performed with the following set of primers:

STF28MCF-2:

(SEQ ID NO.: 33)

5′GGAGCGCCGGTGGATCCTGCTAGCCCATGGACCGAAAACCCGCTGCAG
AAAATTGATGCCGCGCT 3′.
STF28ECR-2:

(SEQ ID NO.: 34)

5′TCTGCAGAATTCACGTAACAGAGACAGCACGTTCTGCGGGACGTCCCG
CAGAACGTGCTGTCTCTGTTACGTGAATTCTGCAGA 3′.

The two reactions were combined in third PCR using STF28BGF1 and STF28ECR-2 to generate STF ˜0.8 kb fragment that was cloned by compatible ends to the Drosophila expression vector pMT/BiP/v5-His digested with BgiII and EcoRI, resulting in the amino acid linker of SEQ ID NO: 19.

### Example 7

### Enhancement of B and T Cell Immunogenicity

### Introduction

Fusion proteins, including fusion proteins comprising a pathogen-associated molecular pattern (e.g., flagellin) and at least two distinct Listeria monocytogenes antigens (referred to herein as “STF2.LIST”) induce enhanced T and B cell responses in manner that is not dependant on a classical CD4-mediated T help (Th) manner. Immunization with STF2.OVA induces significantly enhanced and faster antigen-specific antibody and CD8 T cells responses than observed following immunization with antigen alone or delivered unlinked (also referred to herein as “fused”) with flagellin. The enhanced antigen-specific antibody response includes IgG class switching in the absence of detectable antigen-specific CD4 T helper cells, which is believed to be mediated by a mechanism of B cell activation. Immunization STF2.LIST in the absence of adjuvant induces antigen-specific CD8 T cell responses that are comparable to those observed following a natural infection with live virulent L. monocytogenes. Immunization with STF2.LIST induces immuno-protection in mice following a challenge with live L. monocytogenes in vivo. The compositions described herein may be employed to elicit an enhanced immune response to antigens in situations where CD4 T helper cell responses are impaired or absent, such as in conditions where CD4 T cells responses are suboptimal, impaired or absent, as in, for example, HIV and Hepatitis C infection.

The development of antigen-specific T and B cell responses routinely depend upon the recognition of MHC class II restricted epitopes in the target antigen. Recognition of specific antigen-derived epitopes results in the activation of CD4+T helper (Th) cells that provide critical factors and/or signals necessary for the successful generation of mature B cell responses in vivo. This fact is particularly true for protein targets where the generation of mature antibody responses that are almost entirely T cell dependant.

The results described herein, demonstrate immune responses induced following immunization with recombinant fusion proteins incorporating the TLR5-ligand flagellin. Immunization with STF2.OVA (SEQ ID NO: 51, encoded by SEQ ID NO: 50) or STF2.LIST (SEQ ID NO: 14, encoded by SEQ ID NO: 13) induced enhanced antibody responses and CD8 T cell responses in vivo. In particular, antibody responses were characterized by the rapid induction of IgG despite little to no detectable T helper 2 (Th2) responses to the antigen. Likewise, immunization with STF2.OVA and STF2.LIST induced significantly higher antigen-specific CD8 T cell responses compared to when antigen and flagellin were co-delivered unlinked. These data demonstrate that compositions comprising a pathogen-associated molecular pattern, such as a flagellin, and an antigen, such as at least two distinct Listeria monocytogenes antigens that are fused elicit enhanced antigen-specific antibody and CD8 T cell responses independent of antigen-derived T helper epitopes. Such compositions may be used in the development of treatments, for example, as vaccines, that are not dependant on the presence of classical CD4 T helper responses to the antigen of interest.

### Materials, Methods and Results

Induction of antigen-specific T and B cell responses to STF2.OVA: The development of antigen-specific T and B cell responses following immunization with STF2.OVA was evaluated in C57BL/6 mice. C57BL/6 mice were immunized with PBS, STF2.OVA, or equivalent doses of OVA and STF2 in cocktail on days 0 and 14 with PBS, 25 μg of STF2.OVA or eqimolar equivalents of OVA (12 μg) delivered mixed as individual components and not components of a fusion protein. Some mice were sacrificed before day 14 and examined for antigen-specific B and T cell responses by ELISA and ELISPOT assays, respectively.

In T-cell ELISPOT analysis, CD4 or CD8 cells were purified and restimulated with antigen presented by naive syngeneic APCs. CD4 were stimulated with OVA in IL-5 ELISPOT, while CD8 T cells were stimulated with the OVA-derived peptide SIINFEKL in IFNγ ELISPOTs. A second cohort received a second immunization on day 14, identical to the first, and B and T cell responses were similarly evaluated one week post-boost. Serum OVA-specific IgG1, IgG2a, and IgG2b were examined by ELISA.

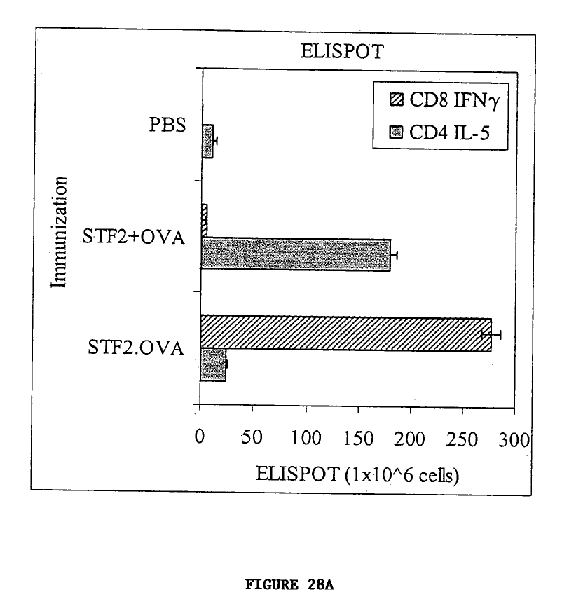

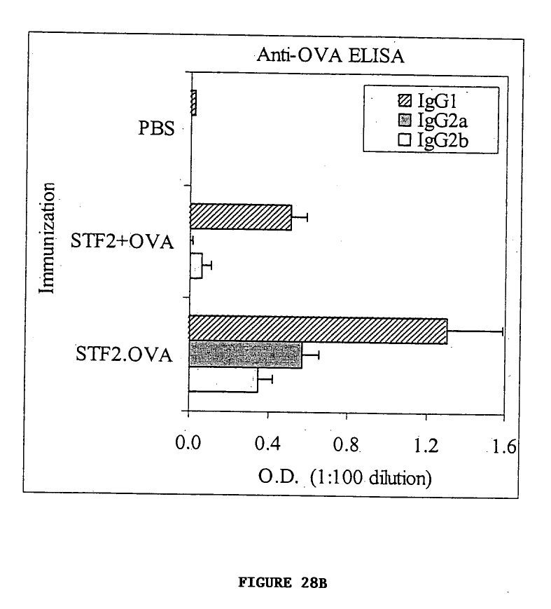

The data depicted in FIGS. 28A and 28B show that mice immunized with OVA mixed with STF2 only mount a CD4 IL-5 response, little to no CD8 IFNγ response, and low IgG1 with no IgG2 titers. By contrast, mice immunized with the STF2.OVA fusion protein mount a weak CD4 IL-5 response, a strong CD8 IFNγ response, and higher antibody titers including both IgG2a and IgG2b. These results suggest that compositions comprising pathogen-associated molecular patterns, such as flagellin, fused to an antigen convert a Th2-biased antigenic response (IL-5, IgG1) to a Th1-biased response with an antibody isotype switching (IgG2a and IgG2b) and effector T cell responses (CD8 IFNγ). Compositions comprising such fusion protein may provide immunization bypass or reduce the requirement for T cell help in generating antigen-specific antibody responses.

To assess whether responses to flagellin fusion proteins may impart protective immunity, challenge studies using the gram positive bacteria Listeria monocytogenes were performed. Protective immunity to this intracellular pathogen is primarily attributed to the CD8 T cell response. Immunity to L. monocytogenes is evident by reduced bacterial burden in the spleens and liver following an i.v. challenge with virulent L. monocytogenes. Using this model, the ability of STF2.OVA to induce protective immunity was examined in C57BL/6 mice.

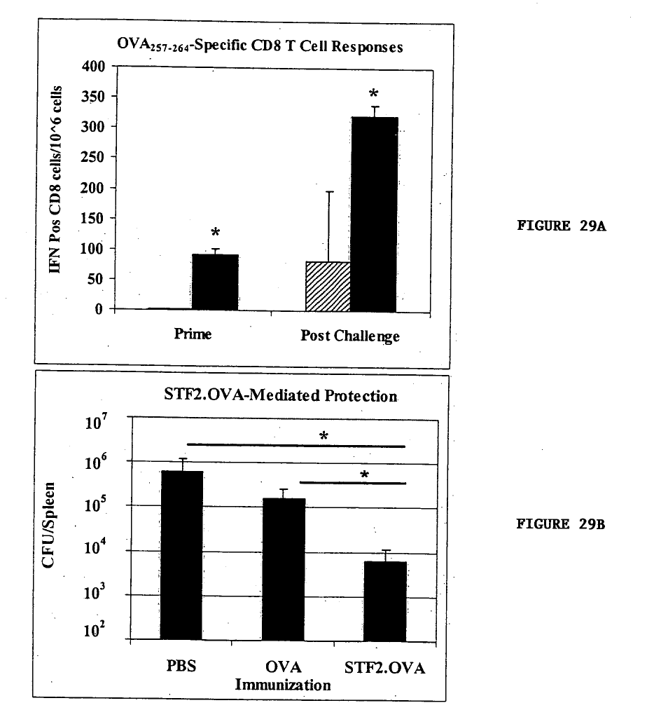

All mice were immunized with the equivalent of 12 μg OVA as OVA alone or STF2.OVA. Three weeks following immunization, mice were challenged i.v. with 2×10⁴ CFU of the OVA-expressing recombinant L. monocytogenes strain JJL-OVA. Antigen-specific CD8 T cell responses were examined in IFNγ ELISPOT assays before and after challenge (day 9 post prime and 5 days post-challenge, respectively) (FIG. 29a). Mice immunized with STF2.OVA have higher antigen-specific IFNα CD8 T cell responses than animals immunized with STF2+OVA following both prime alone (88.3/106 vs. 0.42/106) and post-challenge (310.8/106 vs. 65.2/106).

Thus, a potent CD8 T cell response to the surrogate antigen OVA was induced by immunization with STF2.OVA, as described above.

Immunized mice were challenged i.v. on day 21 with 1×10⁴ CFU of OVA-expressing L. monocytogenes JJL-OVA and bacterial burden in the spleen was examined three days post challenge. Animals immunized with OVA alone developed bacterial bacterial burden in the spleen (1.5×10⁵ CFU) that was not significantly lower than that observed in PBS-immunized (naïve) mice (FIG. 29B). In contrast, STF2.OVA immunized mice exhibited significantly lower bacterial burden (8×10² CFU, P=0.0014) in the spleen compared to naïve mice. These results demonstrate that immunization with STF2.OVA primes robust antigen-specific and protective CD8 T cell responses in vivo.

FIGS. 29A and 29B depict STF2.OVA mediated protection from bacterial challenge in vivo. C57BL/6 mice were immunized s.c. with OVA (12 μg, hatched bars) or STF2.OVA (25 μg, solid bars) or PBS. FIG. 29A: Nine days later, CD8 T cells were isolated and restimulated in vitro with the OVA-derived MHC Class I restricted peptides SIINFEKL in IFNα ELISPOT assays. FIG. 29B: On day 21 post immunization, a second cohort of mice was challenged i.v. with 1×10⁴ CFU of recombinant OVA-expressing L. monocytogenes. Bacterial burden in the spleen was measured three days post-challenge. Data depict the mean±SD of 10 mice per group. The data are representative of results from three experiments with similar results (*P<0.05 by paired Student's t-Test).

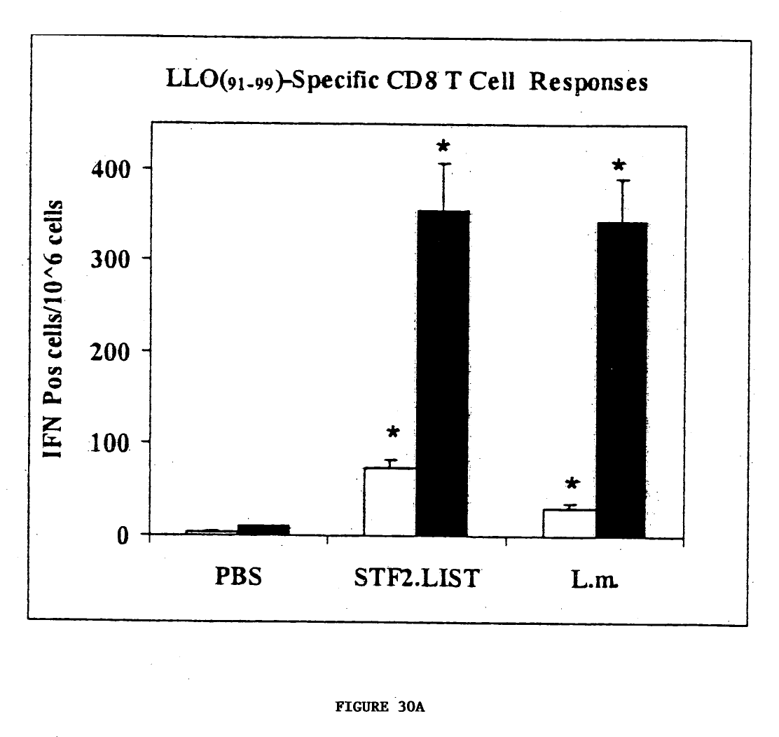

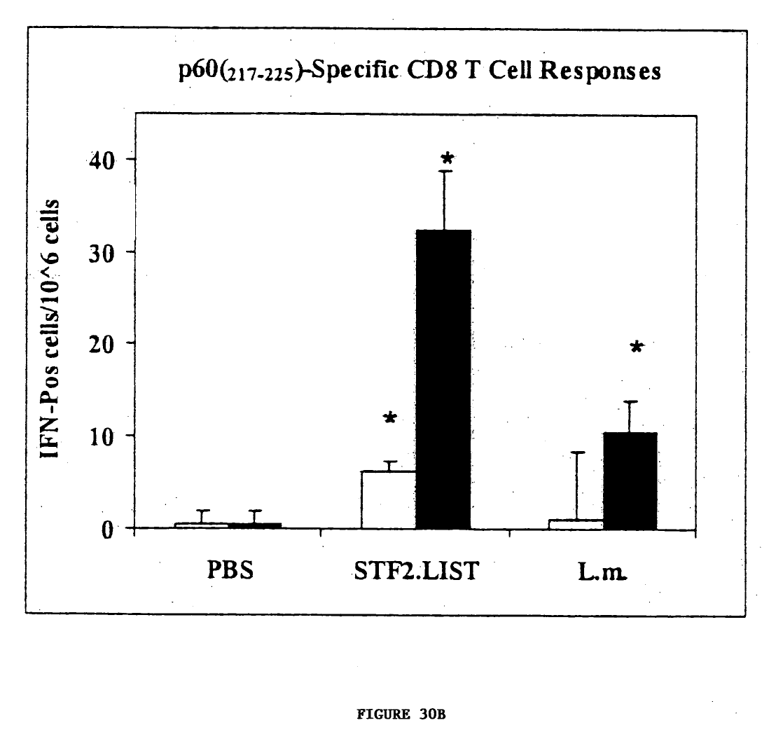

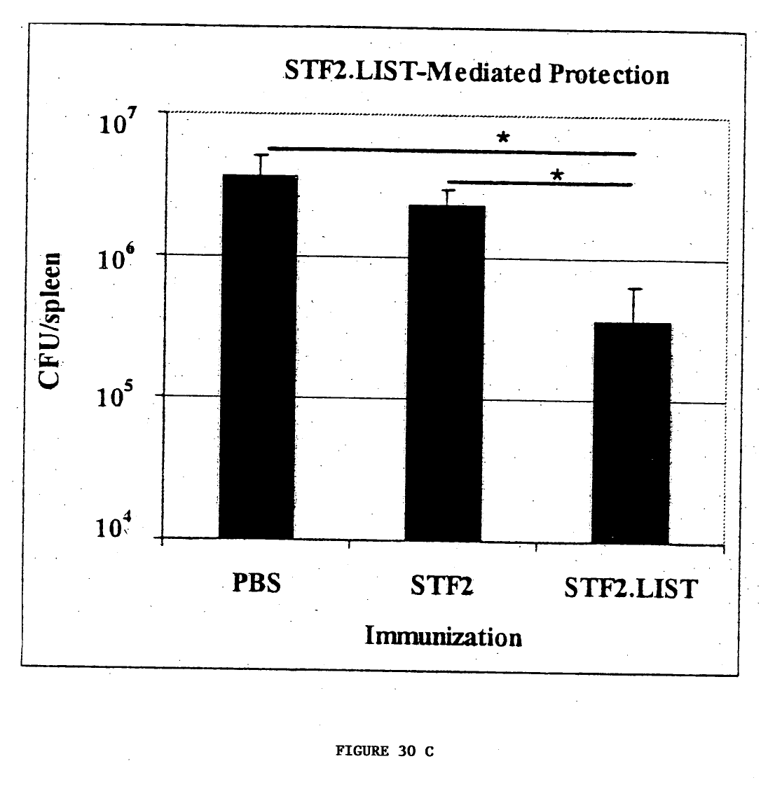

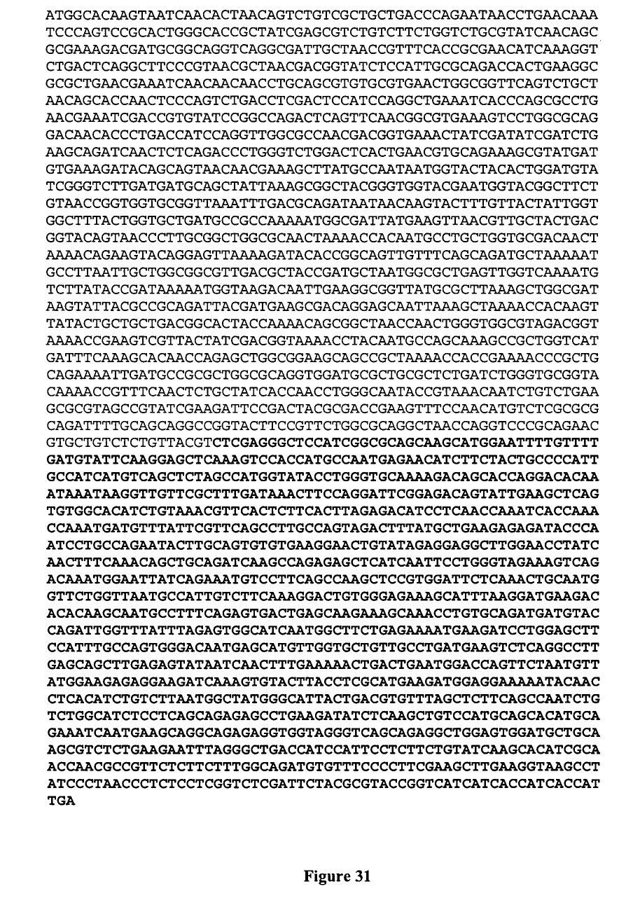

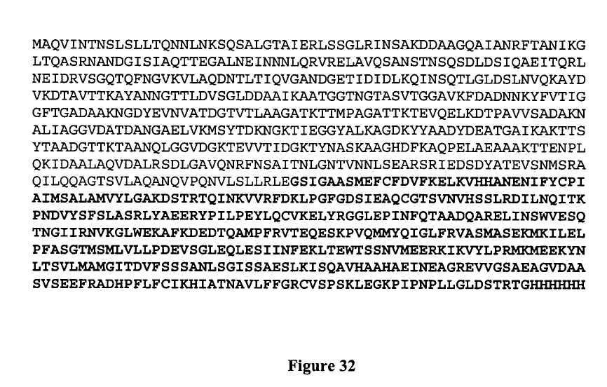

Induction of antigen-specific T and B cell responses to STF2.LIST: The development of antigen-specific T and B cell responses following immunization with STF2.LIST was evaluated in BALB/c mice. Animals were immunized s.c. with PBS or 25 μg of STF2.LIST. As a positive control an additional cohort received a sublethal immunization with 2×10³ CFU of Listeria monocytogenes. Antigen-specific CD8 T cell responses were then examined 9 days later. In T-cell ELISPOT analysis, CD8 cells were purified and restimulated with antigen presented by naive syngeneic APCs. Purified CD8 T cells were stimulated with the LIST-derived LLO91-99 (FIG. 30A) and p60₂₁₇₋₂₂₅ peptides in IFNγ ELISPOT assays, respectively (FIGS. 30A, 30B). In order to verify successful priming to the natural pathogen a replicate cohort was similarly evaluated for antigen specific responses 3 days following a challenged with 2×10⁴ CFU of L. monocytogenes on day 21. The results demonstrate that immunization with STF2.LIST induces antigen-specific CD8 T cell responses that are equal to or greater than those observed to the natural pathogen in vivo.

Next antigen-specific protection in vivo was examined in BALB/c mice following a single immunization with PBS, STF2 (12 μg) or STF2.LIST (25 μg). On day 21 post immunization animals were challenged i.v. with 2×10⁴ CFU of L. monocytogenes. Bacterial burden in the spleen was evaluated three days post-challenge (FIG. 30C). Collectively these results demonstrate that a single immunization with STF2.LIST induces significant numbers of antigen-specific CD8 T cells in vivo and that these CD8 T cells provide protection to a lethal challenge with live pathogen in vivo.

### CONCLUSIONS

These data show that immunization with recombinant flagellin fusion proteins (STF2.OVA and STF2.LIST) induce enhanced T and B cell responses via a novel mechanism that is not dependant on classical CD4-mediated T help (Th). Immunization with STF2.OVA induces significantly enhanced and faster antigen-specific antibody and CD8 T cells responses than observed following immunization with antigen alone or delivered unlinked with flagellin. The enhanced antigen-specific antibody response includes IgG class switching in the absence of detectable antigen-specific CD4 T helper cells, suggesting unique mechanism of B cell activation. Immunization STF2.LIST in the absence of adjuvant induces antigen-specific CD8 T cell responses that are comparable to those observed following a natural infection with live virulent L. monocytogenes. Immunization with STF2.LIST induces immuno-protection in mice following a challenge with live L. monocytogenes in vivo.

Fusion proteins comprising pathogen-associated molecular patterns, such as flagellin, may be useful, alone or in combination with well-known adjuvants, as compositions to stimulate protective immunity in a subject, such as a use in a vaccine to prevent infection to an antigen or for treatment of infections that are a consequence of antigens. Such compositions may be useful in eliciting or stimulating enhanced immune responses to dependant antigens in situations where CD4 T helper responses are impaired or absent, such as in HIV.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

While this invention has been particularly shown and described with references to preferred embodiments thereof, it will be understood by those skilled in the art that various changes in form and details may be made therein without departing from the scope of the invention encompassed by the appended claims.

What is claimed is:

1. A composition comprising:
a) a pathogen associated molecular pattern that activates at least one member selected from the group consisting of TLR2 and TLR5; and
b) at least two distinct Listeria monocytogenes antigens.

2. The composition of claim 1, wherein the pathogen associated molecular pattern and Listeria monocytogenes antigens are components of a fusion protein.

3. The composition of claim 2, wherein the pathogen associated molecular pattern activates a TLR2 signaling pathway.

4. The composition of claim 3, wherein the pathogen associated molecular pattern is at least one member selected from the group consisting of BLP and OmpA.

5. The composition of claim 4, wherein the BLP includes E. coli BLP.

6. The composition of claim 5, wherein the E. coli BLP includes at least a fragment of SEQ ID NO: 1.

7. The composition of claim 6, wherein the fragment of SEQ ID NO: 1 includes the amino acid sequence cysteine-serine-serine-asparagine (CSSN).

8. The composition of claim 4, wherein the OmpA includes E. coli OmpA.

9. The composition of claim 8, wherein the E. coli OmpA includes at least a fragment of SEQ ID NO: 3.

10. The composition of claim 2, wherein the pathogen associated molecular pattern includes at least a fragment of SEQ ID NO: 1 and the Lysteria monocytogenes antigens include at least a fragment of each of SEQ ID NO: 7 and SEQ ID NO: 8.

11. The composition of claim 2, wherein the pathogen associated molecular pattern activates a TLR5 signaling pathway.

12. The composition of claim 11, wherein the pathogen associated molecular pattern is at least a fragment of a flagellin.

13. The composition of claim 12, wherein the flagellin includes a polypeptide selected from the group consisting of H. pylori, V. cholera, S. marcesens, S. flexneri, T. pallidum, L. pneumophilia; B. burgdorferei; C. difficile, R. meliloti, A. tumefaciens; R. lupine; B. clarridgeiae, P. mirabilis, B. subtilus, L. monocytogenes, P. aeruginosa and E. coli.

14. The composition of claim 12, wherein the flagellin is selected from the group consisting of S. typhimurium fljB and E. coli FliC.

15. The composition of claim 14, wherein the S. typhimurium fljB includes at least a fragment of SEQ ID NO: 5.

16. The composition of claim 2, wherein the pathogen associated molecular pattern is a fragment selected from at least one member of the group consisting of the Oprl protein of P. aeruginosa; the fibril subunit protein of Porphyromonas gingivalis; the macrophagic activating lipopeptide 2 (MALP-2) of Mycoplasma fermentans; and p19 from Mycobacterium tuberculosis.

17. The composition of claim 2, wherein the pathogen associated molecular pattern is at least one member selected from the group consisting of lipopolysaccharides; phosphatidyl choline; glucans; peptidoglycans; teichoic acids; lipoteichoic acids; proteins; lipoproteins; lipopeptides; outer membrane proteins (OMPs), outer surface proteins (OSPs); protein components of bacterial cell walls; flagellins; bacterial DNAs; single and double-stranded viral RNAs; unmethylated CpG-DNAs; mannans; mycobacterial membranes; and porins.

18. The composition of claim 2, wherein the antigens include at least a fragment of each of SEQ ID NO: 7 and SEQ ID NO: 8.

19. The composition of claim 2, wherein the antigens are encoded by the nucleic acid sequences that includes a fragment of at least one of SEQ ID NO: 9 and SEQ ID NO: 10.

20. The composition of claim 2, wherein the pathogen associated molecular pattern includes at least a fragment of SEQ ID NO: 3 and the Listeria monocytogenes antigens include at least a fragment of each of SEQ ID NO: 7 and SEQ ID NO: 8.

21. The composition of claim 2, wherein the pathogen associated molecular pattern includes at least a fragment of SEQ ID NO: 5 and the antigens include at least a fragment of each of SEQ ID NO: 7 and SEQ ID NO: 8.

22. The composition of claim 2, wherein the pathogen associated molecular pattern includes at least a fragment of SEQ ID NO: 1 and the antigens include at least a fragment of each of SEQ ID NO: 7 and SEQ ID NO: 8.

23. A composition comprising SEQ ID NO: 12.

24. A composition comprising SEQ ID NO: 16.

25. A composition encoded by a nucleic acid sequence comprising SEQ ID NO: 11.

26. A composition comprising SEQ ID NO: 14.

27. A composition encoded by a nucleic acid sequence comprising SEQ ID NO: 13.

28. A composition comprising:
a) a pathogen associated molecular pattern that activates at least one member selected from the group consisting of TLR2 and TLR5; and
b) a Listeria monocytogenes antigen that is not listeriolysin.

29. The composition of claim 28, wherein the pathogen associated molecular pattern and the Listeria monocytogenes antigen are components of a fusion protein.

30. A composition comprising:
a) a pathogen associated molecular pattern that activates at least one member selected from the group consisting of TLR2 and TLR5; and
b) a Listeria monocytogenes p60 antigen.

31. The composition of claim 30, wherein the pathogen associated molecular pattern and the Listeria monocytogenes p60 antigen are components of a fusion protein.

32. The composition of claim 30, further including at least one additional Listeria monocytogenes antigen.

33. The composition of claim 32, wherein the additional Listeria monocytogenes antigen is listeriolysin.

34. A nucleic acid construct encoding:
a) a pathogen associated molecular pattern that activates at least one member selected from the group consisting of TLR2 and TLR5; and
b) at least two distinct Listeria monocytogenes antigens.

35. A nucleic acid construct of claim 34, wherein the pathogen associated molecular pattern and the Listeria monocytogenes antigens are components of a fusion protein.

36. A nucleic acid construct claim 35, wherein the pathogen associated molecular pattern activates a TLR2 signaling pathway.

37. A nucleic acid construct of claim 36, wherein the pathogen associated molecular pattern is at least one member selected from the group consisting of BLP and OmpA.

38. A nucleic acid construct of claim 37, wherein the BLP includes E. coli BLP.

39. A nucleic acid construct of claim 38, wherein the E. coli BLP includes at least a fragment of SEQ ID NO: 1.

40. A nucleic acid construct of claim 39, wherein the fragment of SEQ ID NO: 1 includes the amino acid sequence cysteine-serine-serine-asparagine (CSSN).

41. A nucleic acid construct of claim 37, wherein the OmpA includes E. coli OmpA.

42. A nucleic acid construct of claim 41, wherein the E. coli OmpA includes at least a fragment of SEQ ID NO: 3.

43. A nucleic acid construct of claim 35, wherein the pathogen associated molecular pattern includes at least a fragment of SEQ ID NO: 1 and the Lysteria monocytogenes antigens include at least a fragment of each of SEQ ID NO: 7 and SEQ ID NO: 8.

44. A nucleic acid construct of claim 35, wherein the pathogen associated molecular pattern activates a TLR5 signaling pathway.

45. A nucleic acid construct of claim 44, wherein the pathogen associated molecular pattern is at least a fragment of a flagellin.

46. A nucleic acid construct of claim 45, wherein the flagellin includes a polypeptide selected from the group consisting of H. pylori, V. cholera, S. marcesens, S. flexneri, T. pallidum, L. pneumophilia; B. burgdorferei; C. difficile, R. meliloti, A. tumefaciens; R. lupine; B. clarridgeiae, P. mirabilis, B. subtilus, L. monocytogenes, P. aeruginosa and E. coli.

47. A nucleic acid construct of claim 45, wherein the flagellin is selected from the group consisting of S. typhimurium fljB and E. coli FliC.

48. A nucleic acid construct of claim 47, wherein the S. typhimurium fljB includes at least a fragment of SEQ ID NO: 5.

49. A nucleic acid construct of claim 35, wherein the pathogen associated molecular pattern is a fragment selected from at least one member of the group consisting of the Oprl protein of P. aeruginosa; the fibril subunit protein of Porphyromonas gingivalis; the macrophagic activating lipopeptide 2 (MALP-2) of Mycoplasma fermentans; and p19 from Mycobacterium tuberculosis.

50. A nucleic acid construct of claim 35, wherein the pathogen associated molecular pattern is at least one member selected from the group consisting of lipopolysaccharides; phosphatidyl choline; glucans; peptidoglycans; teichoic acids; lipoteichoic acids; proteins; lipoproteins; lipopeptides; outer membrane proteins (OMPs), outer surface proteins (OSPs); protein components of bacterial cell walls; flagellins; bacterial DNAs; single and double-stranded viral RNAs; unmethylated CpG-DNAs; mannans; mycobacterial membranes; and porins.

51. A nucleic acid construct of claim 35, wherein the antigens include at least a fragment of each of SEQ ID NO: 7 and SEQ ID NO: 8.

52. A nucleic acid construct of claim 35, wherein the antigens are encoded by the nucleic acid sequences that includes a subsequence of at least one of SEQ ID NO: 9 and SEQ ID NO: 10.

53. A nucleic acid construct of claim 35, wherein the pathogen associated molecular pattern includes at least a fragment of SEQ ID NO: 3 and the Listeria monocytogenes antigens include at least a fragment of each of SEQ ID NO: 7 and SEQ ID NO: 8.

54. A nucleic acid construct of claim 35, wherein the pathogen associated molecular pattern includes at least a fragment of SEQ ID NO: 5 and the antigens include at least a fragment of each of SEQ ID NO: 7 and SEQ ID NO: 8.

55. A nucleic acid construct of claim 35, wherein the pathogen associated molecular pattern includes at least a fragment of SEQ ID NO: 1 and the antigens include at least a fragment of each of SEQ ID NO: 7 and SEQ ID NO: 8.

56. A nucleic acid construct encoding SEQ ID NO: 12.

57. A nucleic acid construct encoding SEQ ID NO: 16.

58. A nucleic acid construct comprising SEQ ID NO: 11.

59. A nucleic acid construct encoding SEQ ID NO: 14.

60. A nucleic acid construct comprising SEQ ID NO: 13.

61. A nucleic acid construct encoding:
a) a pathogen associated molecular pattern that activates at least one member selected from the group consisting of TLR2 and TLR5; and
b) a Listeria monocytogenes antigen that is not listeriolysin.

62. A nucleic acid construct of claim 61, wherein the pathogen associated molecular pattern and the Listeria monocytogenes antigens are components of a fusion protein.

63. A nucleic acid construct encoding:
a) a pathogen associated molecular pattern that activates at least one member selected from the group consisting of TLR2 and TLR5; and
b) a Listeria monocytogenes p60 antigen.

64. A nucleic construct of claim 63, wherein the pathogen associated molecular pattern and the Listeria monocytogenes p60 antigen are components of a fusion protein.

65. A nucleic construct of claim 63, further including at least one additional Listeria monocytogenes antigen.

66. A nucleic construct of claim 65, wherein the additional Listeria monocytogenes antigen is listeriolysin.

67. A vector comprising the nucleic acid construct of claim 34.

68. A vector comprising the nucleic acid construct of claim 61.

69. A vector comprising the nucleic acid construct of claim 63.

70. A host cell comprising the vector of any of claims 67-69.

71. A method of producing a fusion protein, comprising the steps of
a) culturing a host cell comprising a vector, wherein the vector comprises a nucleic acid construct encoding a fusion protein including
i) a pathogen associated molecular pattern that activates at least one member selected from the group consisting of TLR2 and TLR5; and
ii) at least two distinct Listeria monocytogenes antigens; and
b) isolating the fusion protein produced by the host cell.

72. A method of producing a fusion protein, comprising the steps of
a) culturing a host cell comprising a vector, wherein the vector comprises a nucleic acid construct encoding a fusion protein including
i) a pathogen associated molecular pattern that activates at least one member selected from the group consisting of TLR2 and TLR5; and
ii) a Listeria monocytogenes antigen that is not listeriolysin; and
b) isolating the fusion produced by the host cell.

73. A method of producing a fusion protein, comprising the steps of
a) culturing a host cell comprising a vector, wherein the vector comprises a nucleic acid construct encoding a fusion protein including
i) a pathogen associated molecular pattern that activates at least one member selected from the group consisting of TLR2 and TLR5; and
ii) Listeria monocytogenes p60 antigen; and
b) isolating the fusion protein produced by the host cell.

74. A method of stimulating the immune system in a subject, comprising the step of administering to the subject a composition including:
a) a pathogen associated molecular pattern that activates at least one member selected from the group consisting of TLR2 and TLR5; and
b) at least two distinct Listeria monocytogenes antigens.

75. A method of stimulating the immune system in a subject, comprising the step of administering to the subject a composition including:
a) a pathogen associated molecular pattern that activates at least one member selected from the group consisting of TLR2 and TLR5; and
b) a Listeria monocytogenes antigen that is not listeriolysin.

76. A method of stimulating the immune system in a subject, comprising the step of administering to the subject a composition including:
a) a pathogen associated molecular pattern that activates at least one member selected from the group consisting of TLR2 and TLR5; and
b) a Listeria monocytogenes p60 antigen.


SEQUENCE LISTING (51 sequences)

SEQ ID NO: 1 - protein, 24 aa, Artificial Sequence
  E. coli BLP
MKATKLVLGAVILGSTLLAGCSSN

SEQ ID NO: 2 - DNA, 72 bp, Artificial Sequence
  E. coli BLP
gtgtaatacttgtaacgctacatggagattaactcaatctagagggtattaataatgaaagctactaaactg

SEQ ID NO: 3 - protein, 346 aa, Artificial Sequence
  E. coli OmpA
MKKTAIAIAVALAGFATVAQAAPKDNTWYTGAKLGWSQYHDTGFINNNGPTHENQLGAGAFGGYQVNPYVGFEMGYDWLGRMPYKGSVENGAYKAQGVQLTAKLGYPITDDLDIYTRLGGMVWRADTKSNVYGKNHDTGVSPVFAGGVEYAITPEIATRLEYQWTNNIGDAHTIGTRPDNGMLSLGVSYRFGQGEAAPVVAPAPAPAPEVQTKHFTLKSDVLFNFNKATLKPEGQAALDQLYSQLSNLDPKDGSVVVLGYTDRIGSDAYNQGLSERRAQSVVDYLISKGIPADKISARGMGESNPVTGNTCDNVKQRAALIDCLAPDRRVEIEVKGIKDVVTQPQA

SEQ ID NO: 4 - DNA, 1041 bp, Artificial Sequence
  E. coli OmpA
atgaaaaagacagctatcgcgattgcagtggcactggctggtttcgctaccgtagcgcaggccgctccgaaagataacacctggtacactggtgctaaactgggctggtcccagtaccatgacactggtttcatcaacaacaatggcccgacccatgaaaaccaactgggcgctggtgcttttggtggttaccaggttaacccgtatgttggctttgaaatgggttacgactggttaggtcgtatgccgtacaaaggcagcgttgaaaacggtgcatacaaagctcagggcgttcaactgaccgctaaactgggttacccaatcactgacgacctggacatctacactcgtctgggtggcatggtatggcgtgcagacactaaatccaacgtttatggtaaaaaccacgacaccggcgtttctccggtcttcgctggcggtgttgagtacgcgatcactcctgaaatcgctacccgtctggaataccagtggaccaacaacatcggtgacgcacacaccatcggcactcgtccggacaacggcatgctgagcctgggtgtttcctaccgtttcggtcagggcgaagcagctccagtagttgctccggctccagctccggcaccggaagtacagaccaagcacttcactctgaagtctgacgttctgttcaacttcaacaaagcaaccctgaaaccggaaggtcaggctgctctggatcagctgtacagccagctgagcaacctggatccgaaagacggttccgtagttgttctgggttacaccgaccgcatcggttctgacgcttacaaccagggtctgtccgagcgccgtgctcagtctgttgttgattacctgatctccaaaggtatcccggcagacaagatctccgcacgtggtatgggcgaatccaacccggttactggcaacacctgtgacaacgtgaaacagcgtgctgcactgatcgactgcctggctccggatcgtcgcgtagagatcgaagttaaaggtatcaaagacgttgtaactcagccgcaggcttaa

SEQ ID NO: 5 - protein, 506 aa, Artificial Sequence
  Stf 2
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATDGTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLR

SEQ ID NO: 6 - DNA, 1521 bp, Artificial Sequence
  Stf 2
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagcttatgccaataatggtactacactggatgtatcgggtcttgatgatgcagctattaaagcggctacgggtggtacgaatggtacggcttctgtaaccggtggtgcggttaaatttgacgcagataataacaagtactttgttactattggtggctttactggtgctgatgccgccaaaaatggcgattatgaagttaacgttgctactgacggtacagtaacccttgcggctggcgcaactaagaccacaatgcctgctggtgcgacaactaaaacagaagtacaggagttaaaagatacaccggcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaaccacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattctgcagcaggccggtacttccgttctggcgcaggctaaccaggtcccgcagaacgtgctgtctctgttacgttaa

SEQ ID NO: 7 - protein, 133 aa, Artificial Sequence
  Listeria p60
DQNAVPIAYVDQNATTHAVKSGDTIWALSVKYGVSVQDIMSWNNLSSSSIYVGQKLAIKQTANTATPKAEVKTEAPAAEKQAAPVVKENTNTNTATTEKKETATQQQTAPKAPTEAAKPAPAPSTNTNANKTN

SEQ ID NO: 8 - protein, 332 aa, Artificial Sequence
  Listeria LLO
PIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGV

SEQ ID NO: 9 - DNA, 2046 bp, Artificial Sequence
  Listeria p60
tcgatcatcataattctgtctcattatataacatcctccataccttctattatagaataccataaactcatctggcaattcatttcgagtcacgaagaacggaaaaactgccggtttttatattacaaatgtattaagtttttctattaacaaaaaacaataggtttcccatagcgaaagttgttgattaacgttcacatcccacttacactataaaggtttacccagcagtacatctcaagccctaagaatacacgttcgcttttcaactgttacagaattattacaaatagttggtatagtcctctttagcctttggagttattatctcatcatttgttttttaggtgaaaactgggtaaacttagtattaatcaatataaaattaattctcaaatacttaattacgtactgggattttctgaaaaaagagaggagttttatgaatatgaaaaaagcaactatcgcggctacagctgggattgcggtaacagcatttgctgcgccaacaatcgcatccgcaagcactgtagtagtcgaagctggtgatactctttggggtatcgcacaaagtaaagggactactgttgacgcaattaaaaaagcaaacaatttaacaacagataaaatcgtaccaggtcaaaaattacaagtaaataatgaggttgctgctgctgaaaaaacagagaaatctgttagcgcaacttggttaaacgtccgtagtggcgctggtgttgataacagtattattacgtccatcaaaggtggaacaaaagtaactgttgaaacaaccgaatctaacggctggcacaaaattacttacaacgatggaaaaactggtttcgttaacggtaaatacttaactgacaaagcagtaagcactccagttgcaccaacacaagaagtgaaaaaagaaactactactcaacaagctgcacctgctgcagaaacaaaaactgaagtaaaacaaactacacaagcaactacacctgcgcctaaagtagcagaaacgaaagaaactccagtagtagatcaaaatgctactacacacgctgttaaaagcggtgacactatttgggctttatccgtaaaatacggtgtttctgttcaagacattatgtcatggaataatttatcttcttcttctatttatgtaggtcaaaagcttgctattaaacaaactgctaacacagctactccaaaagcagaagtgaaaacggaagctccagcagctgaaaaacaagcagctccagtagttaaagaaaatactaacacaaatactgctactacagagaaaaaagaaacagcaacgcaacaacaaacagcacctaaagcaccaacagaagctgcaaaaccagctcctgcaccatctacaaacacaaatgctaataaaacaaatacaaatacaaatacaaatacaaatacaaacaatactaatacaaatacaccatctaaaaatactaatacaaactcaaatactaatacgaatacaaactcaaatacgaatgctaatcaaggttcttccaacaataacagcaattcaagtgcaagtgctattattgctgaagctcaaaaacaccttggaaaagcttattcatggggtggtaacggaccaactacatttgattgctctggttacactaaatatgtatttgctaaagcgggaatctcccttccacgtacttctggcgcacaatacgctagcactacaagaatctctgaatctcaagcaaaacctggtgatttagtattctttgactatggtagcggaatttctcacgttggtatctacgttggtaatggtcaaatgattaacgcgcaagacaatggcgttaaatacgataacatccacggctctggctggggtaaatatctagttggcttcggtcgcgtataattaataacttaaagtaacctgtggagcaagcagttcgcttcacaggttttttgttggaaattttatcttaatgaaagacggtgtatgatgaagaatctagtaaaagtaaaagttttcctaagttcacaaaaggctataaggaaggttat

SEQ ID NO: 10 - DNA, 1590 bp, Artificial Sequence
  Listeria LLO
atgaaaaaaataatgctagtttttattacacttatattaattagtctaccaatagcacaacaaactgaagcgaaggacgcatctgcatttcataaagaagatttaatttcatccatggcaccaccaacatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatatcgttgtggagaaaaagaagaaatccatcaatcaaaataatgcagacatccaagttgtaaatgcaatttcgagcctaacatatccaggtgctctcgtaaaagcgaattcggaattagtagaaaatcaaccagatgttctccctgtaaaacgtgattcattaacacttagcatcgatttgccaggaatgactaatcaagacaataaaatcgttgtaaaaaatgctactaaatcgaatgttaacaacgcagtaaatacattagtggaaagatggaatgaaaaatatgctcaagcttatccgaatgtaagtgcaaaaattgattatgatgacgaaatggcttacagtgaatcacaattaattgcaaaatttggtactgcatttaaagctgtaaataatagtttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtaaatgcagaaaatcctcctgcatatatctcaagtgtggcatacggccgtcaagtttatttgaaattatcgactaattcccatagtactaaagtaaaagctgcttttgatgctgccgtaagtgggaaatctgtctcaggtgatgtagaattaacaaatatcatcaaaaattcttccttcaaagccgtaatttacggtggttccgcaaaagatgaagttcaaatcatcgatggcaacctcggagacttacgagatattttgaaaaaaggtgctacttttaatcgagaaacaccaggagttcccattgcttatacaacaaatttcttaaaagacaatgaattagctgttattaaaaacaactcagaatatattgaaacaacttcaaaagcttatacagatggaaaaattaatattgatcactctggaggctacgttgctcaattcaacatctcttgggatgaaataaattatgatcctgaaggtaacgaaattgttcaacataaaaactggagcgaaaacaataaaagcaagctagctcatttcacatcgtccatctatttgccaggtaacgcaagaaatattaatgtttacgccaaagaatgcactggtttagcttgggaatggtggagaacggtaattgatgaccggaacttaccacttgtgaaaaatagaaatatctccatctggggcactacgctttatccgaaatatagtaatagtgtagataatccaatcgaataa

SEQ ID NO: 11 - DNA, 1503 bp, Artificial Sequence
  P2. List
atgaaagctactaaactggtactgggcgcggtaatcctgggttctactctgctggcaggttgctccagcaacgaattcccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaactcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtaaatgcagaaaatcctcctgcatatatctcaagtgtggcatacggccgtcaagtttatttgaaattatcgactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagtagatcaaaatgctgttcccattgcttatgtagatcaaaatgctactacacacgctgtcaaaagcggtgacactatttgggctttatccgtaaaatacggtgtttctgttcaagacattatgtcatggaataatttatcttcttcttctatttatgtaggtcaaaagcttgctattaaacaaactgctaacacagctactccaaaagcagaagtgaaaacggaagctccagcagctgaaaaacaagcagctccagtagttaaagaaaatactaacacaaatactgctactacagagaaaaaagaaacagcaacgcaacaacaaacagcacctaaagcaccaacagaagctgcaaaaccagctcctgcaccatctacaaacacaaatgctaataaaacaaatacactcgagcaccaccaccaccaccactga

SEQ ID NO: 12 - protein, 500 aa, Artificial Sequence
  P2. List
MKATKLVLGAVILGSTLLAGCSSNEFPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVDQNAVPIAYVDQNATTHAVKSGDTIWALSVKYGVSVQDIMSWNNLSSSSIYVGQKLAIKQTANTATPKAEVKTEAPAAEKQAAPVVKENTNTNTATTEKKETATQQQTAPKAPTEAAKPAPAPSTNTNANKTNTLEHHHHHH

SEQ ID NO: 13 - DNA, 2613 bp, Artificial Sequence
  Stf2/LLO-p60
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagcttatgccaataatggtactacactggatgtatcgggtcttgatgatgcagctattaaagcggctacgggtggtacgaatggtacggcttctgtaaccggtggtgcggttaaatttgacgcagataataacaagtactttgttactattggtggctttactggtgctgatgccgccaaaaatggcgattatgattacgatgaagcgacaggagcaattaaagctaaaaccacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattctgcagcaggccggtacttccgttctggcgcaggctaaccaggtcccgcagaacgtgctgtctctgttacgtgaattcccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaactcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtaaatgcagaaaatcctcctgcatatatctcaagtgtggcatacggccgtcaagtttatttgaaattatcgactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagtagatcaaaatgctgttcccattgcttatgtagatcaaaatgctactacacacgctgtcaaaagcggtgacactatttgggctttatccgtaaaatacggtgtttctgttcaagacattatgtcatggaataatttatcttcttcttctatttatgtaggtcaaaagcttgctattaaacaaactgctaacacagctactccaaaagcagaagtgaaaaatactgctactacagagaaaaaagaaacagcaacgcaacaacaaacagcacctaaagcaccaacagaagctgcaaaaccagctcctgcaccatctacaaacacaaatgctaataaaacaaatacactcgagcaccaccaccaccaccactga

SEQ ID NO: 14 - protein, 870 aa, Artificial Sequence
  Stf2 and LLO-p60
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYDYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLREFPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVDQNAVPIAYVDQNATTHAVKSGDTIWALSVKYGVSVQDIMSWNNLSSSSIYVGQKLAIKQTANTATPKAEVKNTATTEKKETATQQQTAPKAPTEAAKPAPAPSTNTNANKTNTLEHHHHHH

SEQ ID NO: 15 - DNA, 1443 bp, Artificial Sequence
  P2 and LLO-p60
tgctccagcaacgaattcccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaactcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtaaatgcagaaaatcctcctgcatatatctcaagtgtggcatacggccgtcaagtttatttgaaattatcgactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagtagatcaaaatgctgttcccattgcttatgtagatcaaaatgctactacacacgctgtcaaaagcggtgacactatttgggctttatccgtaaaatacggtgtttctgttcaagacattatgtcatggaataatttatcttcttcttctatttatgtaggtcaaaagcttgctattaaacaaactgctaacacagctactccaaaagcagaagtgaaaacggaagctccagcagctgaaaaacaagcagctccagtagttaaagaaaatactaacacaaatactgctactacagagaaaaaagaaacagcaacgcaacaacaaacagcacctaaagcaccaacagaagctgcaaaaccagctcctgcaccatctacaaacacaaatgctaataaaacaaatacactcgagcaccaccaccaccaccactga

SEQ ID NO: 16 - protein, 480 aa, Artificial Sequence
  P2 and LLO-p60
CSSNEFPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVDQNAVPIAYVDQNATTHAVKSGDTIWALSVKYGVSVQDIMSWNNLSSSSIYVGQKLAIKQTANTATPKAEVKTEAPAAEKQAAPVVKENTNTNTATTEKKETATQQQTAPKAPTEAAKPAPAPSTNTNANKTNTLEHHHHHH

SEQ ID NO: 17 - protein, 267 aa, Artificial Sequence
  Truncated Stf2
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQTENPLQKIDAALAQVDALRSDLGAVQNRFNSAITNLGNTVNNLSEARSRIEDSDYATEVSNMSRAQILQQAGTSVLAQANQVPQNVLSLLR

SEQ ID NO: 18 - protein, 277 aa, Artificial Sequence
  Truncated Stf2
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQGAPVDPASPWTENPLQKIDAALAQVDALRSDLGAVQNRFNSAITNLGNTVNNLSEARSRIEDSDYATEVSNMSRAQILQQAGTSVLAQANQVPQNVLSLLR

SEQ ID NO: 19 - protein, 10 aa, Artificial Sequence
  Linker
GAPVDPASPW

SEQ ID NO: 20 - DNA, 30 bp, Artificial Sequence
  Linker
ggagcgccggtggatcctgctagcccatgg

SEQ ID NO: 21 - DNA, 831 bp, Artificial Sequence
  Truncated Stf2
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagggagcgccggtggatcctgctagcccatggaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattctgcagcaggccggtacttccgttctggcgcaggctaaccaggtcccgcagaacgtgctgtctctgttacgt

SEQ ID NO: 22 - DNA, 24 bp, Artificial Sequence
  Primer
atggcacaagtaatcaacactaac

SEQ ID NO: 23 - DNA, 30 bp, Artificial Sequence
  Primer
ctcgagacgtaacagagacagcacgttctg

SEQ ID NO: 24 - DNA, 37 bp, Artificial Sequence
  Primer
aatgaaaaagacagctatcgcgattgcagtggcactg

SEQ ID NO: 25 - DNA, 40 bp, Artificial Sequence
  Primer
aagcttcgaattgcccttagcctgcggctgagttacaacg

SEQ ID NO: 26 - DNA, 37 bp, Artificial Sequence
  Primer
cttaaagaattcccaatcgaaaagaaacacgcggatg

SEQ ID NO: 27 - DNA, 30 bp, Artificial Sequence
  Primer
ttctactaattccgagttcgcttttacgag

SEQ ID NO: 28 - DNA, 30 bp, Artificial Sequence
  Primer
ctcgtaaaagcgaactcggaattagtagaa

SEQ ID NO: 29 - DNA, 37 bp, Artificial Sequence
  Primer
agaggtctcgagtgtatttgttttattagcatttgtg

SEQ ID NO: 30 - protein, 14 aa, Artificial Sequence
  Linker
GKPIPNPLLGLDST

SEQ ID NO: 31 - DNA, 38 bp, Artificial Sequence
  Primer
ctcgggagatctgcacaagtaatcaacactaacagtct

SEQ ID NO: 32 - DNA, 59 bp, Artificial Sequence
  Primer
ccatgggctagcaggatccaccggcgctccctgcacgttcagtgagtccagacccaggg

SEQ ID NO: 33 - DNA, 65 bp, Artificial Sequence
  Primer
ggagcgccggtggatcctgctagcccatggaccgaaaacccgctgcagaaaattgatgccgcgct

SEQ ID NO: 34 - DNA, 84 bp, Artificial Sequence
  Primer
tctgcagaattcacgtaacagagacagcacgttctgcgggacgtcccgcagaacgtgctgtctctgttacgtgaattctgcaga

SEQ ID NO: 35 - protein, 9 aa, Artificial Sequence
  Listeria p60
KYGVSVQDI

SEQ ID NO: 36 - protein, 12 aa, Artificial Sequence
  Listeria p60
TEAAKPAPAPST

SEQ ID NO: 37 - protein, 12 aa, Artificial Sequence
  Listeria LLO
NEKYAQAYPNVS

SEQ ID NO: 38 - protein, 12 aa, Artificial Sequence
  Listeria LLO
AFDAAVSGKSVS

SEQ ID NO: 39 - protein, 9 aa, Artificial Sequence
  Listeria LLO
VAYGRQVYL

SEQ ID NO: 40 - protein, 8 aa, Artificial Sequence
  Listeria LLO
AYGRQVYL

SEQ ID NO: 41 - protein, 12 aa, Artificial Sequence
  Listeria LLO
QIYYNVNVNEPT

SEQ ID NO: 42 - protein, 484 aa, Artificial Sequence
  P2. LIST
MKATKLVLGAVILGSTLLAGCSSNEPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVVDQNATTHAVKSGDTIWALSVKYGVSVQDIMSWNNLSSSSIYVGQKLAIKQTANTATPKAEVKTEAPAAEKQAAPVVKENTNTNTATTEKKETATQQQTAPKAPTEAAKPAPAPSTNTNANKTNTNT

SEQ ID NO: 43 - DNA, 33 bp, Artificial Sequence
  primer
ttagtccatatgaaagctactaaactggtactg

SEQ ID NO: 44 - DNA, 34 bp, Artificial Sequence
  primer
aagattgaattcgcggtatttagtagccatgttg

SEQ ID NO: 45 - DNA, 18 bp, Artificial Sequence
  Primer
accgtcatcaccgaaacg

SEQ ID NO: 46 - DNA, 33 bp, Artificial Sequence
  Primer
aactaagaattcgttgctggagcaacctgccag

SEQ ID NO: 47 - DNA, 35 bp, Artificial Sequence
  Primer
gtctcgaggaattcccaatcgaaaagaaacacgcg

SEQ ID NO: 48 - DNA, 27 bp, Artificial Sequence
  Primer
acggcactggtcaacttggccatggtg

SEQ ID NO: 49 - DNA, 30 bp, Artificial Sequence; identical to SEQ ID NO: 20
  Linker

SEQ ID NO: 50 - protein, 900 aa, Artificial Sequence
  STF2.OVA
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATDGTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLRLEGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPSKLEGKPIPNPLLGLDSTRTGHHHHHH

SEQ ID NO: 51 - DNA, 2763 bp, Artificial Sequence
  STF2.OVA
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagcttatgccaataatggtactacactggatgtatcgggtcttgatgatgcagctattaaagcggctacgggtggtacgaatggtacggcttctgtaaccggtggtgcggttaaatttgacgcagataataacaagtactttgttactattggtggctttactggtgctgatgccgccaaaaatggcgattatgaagttaacgttgctactgacggtacagtaacccttgcggctggcgcaactaaaaccacaatgcctgctggtgcgacaactaaaacagaagtacaggagttaaaagatacaccggcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaaccacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgtctctgttacgtctcgagggctccatcggcgcagcaagcatggaattttgttttgatgtattcaaggagctcaaagtccaccatgccaatgagaacatcttctactgccccattgccatcatgtcagctctagccatggtatacctgggtgcaaaagacagcaccaggacacaaataaataaggttgttcgctttgataaacttccaggattcggagacagtattgaagctcagtgtggcacatctgtaaacgttcactcttcacttagagacatcctcaaccaaatcaccaaaccaaatgatgtttattcgttcagccttgccagtagactttatgctgaagagagatacccaatcctgccagaatacttgcagtgtgtgaaggaactgtatagaggaggcttggaacctatcaactttcaaacagctgcagatcaagccagagagctcatcaattcctgggtagaaagtcagacaaatggaattatcagaaatgtccttcagccaagctccgtggattctcaaactgcaatggttctggttaatgccattgtcttcaaaggactgtgggagaaagcatttaaggatgaagacacacaagcaatgcctttcagagtgactgagcaagaaagcaaacctgtgcagatgatgtaccagattggtttatttagagtggcatcaatggcttctgagaaaatgaagatcctggagcttccatttgccagtgggacaatgagcatgttggtgctgttgcctgatgaagtctcaggccttgagcagcttgagagtataatcaactttgaaaaactgactgaatggaccagttctaatgttatggaagagaggaagatcaaagtgtacttacctcgcatgaagatggaggaaaaatacaacctcacatctgtcttaatggctatgggcattactgacgtgtttagctcttcagccaatctgtctggcatctcctcagcagagagcctgaagatatctcaagctgtccatgcagcacatgcagaaatcaatgaagcaggcagagaggtggtagggtcagcagaggctggagtggatgctgcaagcgtctctgaagaatttagggctgaccatccattcctcttctgtatcaagcacatcgcaaccaacgccgttctcttctttggcagatgtgtttccccttcgaagcttgaaggtaagcctatccctaaccctctcctcggtctcgattctacgcgtaccggtcatcatcaccatcaccattga